(19) United States
(12) Patent Application Publication
(10) Pub. No.: US 2009/0136561 A1
(43) Pub. Date: May 28, 2009

(54) Non-peptidyl agents with pHSP20-like activity, and uses thereof

(75) Inventors: Moritz von Rechenberg, Salt Lake City, UT (US); John M. Peltier, Sandy, UT (US); Sudhir R. Sahasrabudhe, Sandy, UT (US); Srdjan Askovic, Salt Lake City, UT (US); Robert Selliah, Midvale, UT (US); Thomas Zarembinski, Salt Lake City, UT (US)

Correspondence Address: ROPES & GRAY LLP, PATENT DOCKETING 39/41, ONE INTERNATIONAL PLACE, BOSTON, MA, 02110-2624, US

(73) Assignee: Prolexys Pharmaceuticals, Inc., Salt Lake City, UT (US)

(21) Appl. No.: 12/288,458

(22) Filed: Oct. 20, 2008

Related U.S. Application Data
(62) Division of application No. 11/065,270, filed on Feb. 23, 2005, now abandoned.
(60) Provisional application No. 60/547,157, filed on Feb. 23, 2004.

(51) Int. Cl. A61L 27/54; A61K 31/352; A61K 31/47; A61K 31/4409; C12N 5/00; A61P 21/00; C12Q 1/02; C07D 215/12; C07D 311/02; C07D 213/56

(52) U.S. Cl. 424/425; 514/456; 514/313; 514/354; 546/173; 549/404; 546/336; 435/375; 435/29

(57) ABSTRACT

The present invention provides compositions and methods for modulating smooth muscle cells. The present invention also provides methods of identifying small molecule candidate therapeutic agents for modulating smooth muscle.

### RELATED APPLICATION

This application is a divisional of U.S. application Ser. No. 11/065,270, filed Feb. 23, 2005, which claims the benefit of U.S. Provisional Application No. 60/547,157, filed Feb. 23, 2004. The entire teachings of the above applications are incorporated herein by reference.

### BACKGROUND OF THE INVENTION

Vasospasm/vasoconstriction and bronchospasm/bronchoconstriction represent significantly preventable causes of morbidity and death. Smooth muscle (vascular smooth muscle (VSM) and airway smooth muscle (ASM)) is able to maintain tension for extended periods at low energy cost. This is essential for the autonomous and continuous regulation of blood flow to the organs, breathing, etc. However, in diseases associated with vasospasm/vasoconstriction, there is an abnormal contraction of the blood vessels to a vascular bed combined with the blood vessels having a diminished ability to relax. This restricts the blood flow and in consequence the oxygen supply. A variety of vascular beds including cardiac, mesenteric, placental, uterine and cerebral may be affected with consequent serious clinical implications such as organ damage, stroke, death or miscarriage (Rajani et al., 1991, Postgrad. Medical Journal 67:78-80; Gewertz and Zarins, 1991, J. Vasc. Surg. 14:382-385). In diseases associated with bronchospasm/bronchoconstriction, there is an abnormal contraction of the airways in the lung, which can lead to difficulty in breathing.

It is a goal of the present invention to provide methods and compositions effective in the treatment of clinical conditions associated with aberrant regulation of the tone of smooth muscle, especially, but not limited to vasospasm/vasoconstriction and bronchospasm/bronchoconstriction.

### SUMMARY OF THE INVENTION

The present invention provides non-peptidyl small molecules (also referred to herein as “agents”) for modulating one or more biological activities mediated by 14-3-3 proteins, such as 14-3-3γ or 14-3-3η. The compositions of the present invention can be used to induce or inhibit the cellular effects mediated by the binding of phosphorylated HSP proteins, such as phosphorylated HSP20 (herein pHSP20) with 14-3-3 proteins, and/or the biological or the cellular effects mediated by the binding of phosphorylated cofilin (herein pCofilin) with 14-3-3 proteins and/or the cellular effects of pHSP20 that lead to smooth muscle relaxation independent of 14-3-3 proteins. The compositions of the present invention can be used as part of a method to alter smooth muscle tone. In certain embodiments, the subject compositions can be used to induce constriction or dilation, as the case may be, of a tubular tissue structure having smooth muscle lumen.

For instance, the methods and compositions of the subject invention can be used as part of treatments for altering vascular tone (inducing vasoconstriction or vasorelaxation), which include non-peptidyl small molecule agents that bind to 14-3-3 proteins, such as 14-3-3γ or 14-3-3η. These agents may, in certain instances, inhibit the formation of, or reduce the stability of, complexes including phosphorylated HSP proteins (pHSP), such as pHSP20 and thereby prevent the biological consequence of phosphorylation of the HSP. In other instances, the agents mimic the effect of pHSP20 binding to the 14-3-3 protein and cause at least some of the same biological changes induced by phosphorylation of the HSP.

The methods and compositions of the present invention can also be used for inducing changes in bronchial tone, e.g., inducing bronchial contriction or bronchial relaxation. As above, this is accomplished using non-peptidyl small molecule agents that bind to 14-3-3 proteins including, but not limited to, 14-3-3γ or 14-3-3η. Bronchial relaxation can be induced with agents that mimic the effects of pHSP20 binding.

In another aspect of the invention, pHSP20 or a mimetic thereof, such as a fragment, derivative (e.g., PTD-HSP20 peptide) or functional mutant thereof (suitable peptide can fall in more than one of these categories, e.g., a fragment can be derivatized), can be used for inducing changes in bronchial tone, e.g., inducing bronchial contriction or bronchial relaxation. This is typically accomplished using peptides that bind to 14-3-3 proteins including, but not limited to, 14-3-3γ or 14-3-3η, and/or modulate pHSP20/14-3-3 and/or 14-3-3/pCofilin complex formation. Bronchial relaxation can be induced with agents that mimic the effects of pHSP20 binding.

In certain embodiments, the peptidyl or non-peptidyl agent alters formation and/or stability of complexes including phosphorylated HSP20 or phosphorylated cofilin, or mimics the effect of pHSP20 binding on cytoskeletal dynamics (e.g., the effect caused by pHSP20 binding to 14-3-3γ and/or 14-3-3η). When the agent binds to a 14-3-3, the binding can have a K_{d} of 10 μM or less, such as 1 μM or less, for example 100 nM, or even 10 nM or less.

In certain embodiments, the agent selectively binds to 14-3-3γ and/or 14-3-3η, by at least a factor of 2, relative to other 14-3-3 proteins. In certain preferred embodiments, the agent binds to 14-3-3γ and/or 14-3-3η with a K_{d} at least 5 times less than other 14-3-3 proteins (e.g., 14-3-3γ or 14-3-3η, and more preferably with a K_{d} at least 10, 50, 100 or even 1000 times less. Selectivity for a particular 14-3-3 can also, or alternatively, be provided by tissue-localized or directed delivery of the agent. For instance, preferred agents of the present invention do not affect actin or other cytoskeletal structures in non-smooth muscle tissues, such as neurons.

In certain embodiments, the non-peptidyl agent has a molecular weight less than 2000 amu, and even more preferably less than 1500 or even 1000 amu. Preferably the agent is cell-permeable.

In certain embodiments, the agent is itself cell-permeable.

In certain embodiments, the agent is orally active.

In certain embodiments, the agent is a non-peptidyl organic molecule.

In certain embodiments, the non-peptidyl agent induces vasodilation. For example, the agent may promote an actin depolymerizing activity of cofilin. In certain cases, the agent antagonizes formation or stability of complexes including 14-3-3 proteins, such as 14-3-3γ, and cofilin in smooth muscle (e.g., vascular) tissue.

In other embodiments, the non-peptidyl agents induce vasoconstriction. For example, the agent “derepresses” HSP20 inhibition of complexes including 14-3-3 proteins, such as 14-3-3γ or 14-3-3η, and cofilin in smooth muscle (e.g., vascular) tissue. Alternatively, the agent inhibits an actin depolymerizing activity of cofilin.

In still other embodiments, the non-peptidyl agent induces bronchial dilation. For example, the agent may promote an actin depolymerizing activity of cofilin in bronchial tissue. In certain cases, the agent antagonizes formation or stability of complexes including 14-3-3 proteins, such as 14-3-3γ or 14-3-3η, and cofilin. In a particular embodiment, the agent is administered prior to, after and/or with one or more antibacterials, antivirals, antifungals, antihistamines, bronchial dilators, leukotriene receptor antagonists, proteins, enzymes, hormones, nonsteroidal anti-inflammatories, cytokines, and steroids.

The compositions of the present invention may also include one or more pharmaceutical agents, such as immunosuppressive agents, anti-proliferatives, corticosteroids, angiostatic steroids, anti-parasitic drugs, anti-glaucoma drugs, antibiotics, RNAi and antisense compounds, differentiation modulators, antiviral drugs, anticancer drugs, and anti-inflammatory drugs.

Another aspect of the present invention provides a method for altering vasodilatory properties of blood vessels, comprising treating target blood vessels with the compositions of the present invention as described above.

Another aspect of the present invention provides a method for treating a patient suffering from the effects of vasoconstriction or from restricted blood flow, comprising administering the compositions of the present invention as described above, wherein the agent enhances vasodilation.

Another aspect of the present invention provides a method of inducing vasodilation to treat or prevent a vasocontractive response or condition, comprising administering the subject composition as described above, wherein the agent enhances vasodilation. Optionally, the vasocontractive response or condition is selected from the group consisting of: a renal vasoconstrictive disorder (including glomerular disease and chronic renal disease); and a cardiovascular disease (including hypertension, myocardial infarction, and myocardial ischemia). In certain cases, the vasoconstrictive response is a result of production of leukotrienes, such as associated with a medical disorder selected from the group consisting of: asthma, anaphylactic reactions, allergic reactions, shock, inflammation, rheumatoid arthritis, gout, psoriasis, allergic rhinitis, adult respiratory distress syndrome, Crohn's disease, endotoxin shock, traumatic shock, hemmorrhagic shock, bowel ischemic shock, benign prostatic hypertrophy, inflammatory bowel disease, circulatory shock, brain injury, and systemic lupus erythematosus. In a specific embodiment, the vasoconstrictive response is drug induced, for example, by Cyclosporine A (CSA).

Another aspect of the present invention provides a method for treating a patient suffering vasospasms, comprising administering to the subject a composition as described above, where the agent enhances vasodilation.

Another aspect of the present invention provides a method of increasing blood flow in the circulatory system of a mammal comprising administering to said mammal an amount of the subject composition effective to induce vasodilation.

Another aspect of the present invention provides a method for treating erectile dysfunction comprising administering the subject composition, where the agent enhances vasodilation.

Another aspect of the present invention provides a method for inducing vasodilation comprising administering the subject composition, where the agent enhances vasodilation.

Another aspect of the present invention provides a method for inducing vasoconstriction in a patient suffering from the effects of vasodilation or for inhibiting/counteracting vasodilation, comprising administering the subject composition, where the agent inhibits vasodilation. For example, the agent is used to reduce resistance to contractile agonists. In certain cases, the agent is as part of a treatment for hyperthermia and/or sepsis presenting with vasodilatory shock.

In certain embodiments, the composition of the present invention is administered intravenously, orally, nasally, bucally, parenterally, by inhalation, by topical application or transdermally. Alternatively, the agent is administered via local administration. For example, local administration of the composition is via a suture, a vascular implant, a stent, a heart valve, a drug pump, a drug delivery catheter, an infusion catheter, a drug delivery guidewire or an implantable medical device.

In certain embodiments, methods of the present invention are used to treat diseases characterized by abnormal proliferation or migration of smooth muscle cells. In a specific embodiment, methods of the invention are used to treat disease characterized by increased levels of phosphorylated HSP20. In another specific embodiment, methods of the invention are used to treat disease characterized by decreased levels of phosphorylated HSP20 or increased levels of 14-3-3.

In certain embodiments, methods of the present invention are used to treat patients that have undergone, are undergoing, or will undergo a procedure selected from the group consisting of: angioplasty, vascular stent placement, endarterectomy, atherectomy, bypass surgery, vascular grafting, organ transplant, prosthetic implant emplacement (e.g., heart valve replacement), microvascular reconstructions, plastic surgical flap construction, and catheter emplacement.

In certain embodiments, methods of the present invention are used to treat a disease selected from the group consisting of: stenosis, restenosis, atherosclerosis, hypertension, angina, ischemic disease, intimal hyperplasia, coronary vasospasm, coronary ischemia, congestive heart failure or pulmonary edema associated with acute myocardial infarction, thrombosis, stroke, platelet adhesion, platelet aggregation, smooth muscle cell proliferation, vascular complications associated with the use of medical devices, wounds associated with the use of medical devices, myocardial infarction, pulmonary thromboembolism, cerebral thromboembolism, thrombophlebitis, thrombocytopenia or bleeding disorders, bradycardia, asthma (bronchospasm), toxemia of pregnancy, pre-term labor, pre-eclampsia/eclampsia, Raynaud's disease, Raynaud's phenomenon, hemolyticuremia, non-occlusive mesenteric ischemia, anal fissure, achalasia, impotence, migraine, ischemic muscle injury associated with smooth muscle spasm, and vasculopathy.

Another aspect of the invention provides a respiratory formulation that includes a small organic non-peptidyl agent that binds to a 14-3-3 protein and alters formation and/or stability of complexes including phosphorylated heat shock protein 20 (pHSP20), or mimics the effect of pHSP20 binding to the 14-3-3 protein, which agent has a molecular weight less than 2000 amu and a K_{d} for binding 14-3-3γ of 10 μM or less, such as 2.5 nM or less.

Another aspect of the present invention provides a sustained release formulation comprising a polymer matrix and the subject composition dispersed in the polymer. Optionally, the duration of release of the agent from the polymer matrix is at least 24 hours. In a specific embodiment, the polymer is non-bioerodible. Examples of the non-bioerodible polymers include polyurethane, polysilicone, poly(ethylene-co-vinyl acetate), polyvinyl alcohol, and derivatives and copolymers thereof. Alternatively, the polymer is bioerodible. Examples of the bioerodible polymer include polyanhydrides, polylactic acid, polyglycolic acid, polyorthoesters, polyalkylcyanoacrylates, and derivatives and copolymers thereof. In certain cases, the system is adapted to be injected or implanted into a body.

Another aspect of the present invention provides a medical device comprising: (i) a substrate having a surface; and, (ii) a coating adhered to the surface, said coating comprising a polymer matrix having the subject composition dispersed therein in a manner that permits the agent to be eluted from the matrix under physiological conditions. For example, the substrate is a surgical implement selected from a screw, a plate, a washer, a suture, a prosthesis anchor, a tack, a staple, an electrical lead, a valve, and a membrane. To illustrate, the devices of the present invention include, but are not limited to, catheters, implantable vascular access ports, blood storage bags, blood tubing, central venous catheters, arterial catheters, vascular grafts, intraaortic balloon pumps, heart valves, cardiovascular sutures, artificial hearts, a pacemaker, ventricular assist pumps, extracorporeal devices, blood filters, hemodialysis units, hemoperfusion units, plasmapheresis units, and filters adapted for deployment in a blood vessel. In a specific embodiment, the device is a vascular stent. Optionally, the device is an expandable stent, and said coating is flexible to accommodate compressed and expanded states of said expandable stent.

Another aspect of the present invention provides a coated device combination, comprising a medical device for implantation within a patient's body, said medical device having one or more surfaces coated with a polymer formulation including the subject composition in a manner that permits the coated surface to release the agent over a period of time when implanted in the patient.

In certain embodiments, the present invention provides an intraluminal medical device coated with a sustained release system comprising a biologically tolerated polymer and the subject composition dispersed in the polymer, said device having an interior surface and an exterior surface; said device having said system applied to at least a part of the interior surface, the exterior surface, or both.

Another aspect of the present invention provides a coating composition for use in delivering a medicament from the surface of a medical device positioned in vivo, the composition comprising a polymer matrix having an agent that alters formation or stability of complexes including phosphorylated heat shock protein 20 (pHSP20) and a 14-3-3 protein, such as 14-3-3γ or 14-3-3η, or mimics the effect of pHSP20 binding to a 14-3-3 protein, such as 14-3-3γ or 14-3-3η, which coating composition is provided in liquid or suspension form for application to the surface of said medical device by spraying and/or dipping the device in said composition.

Another aspect of the present invention provides a method for regulating contractility and/or tone of explanted vascular tissue, comprising contacting the explanted tissue in vitro with the subject composition.

Another aspect of the present invention provides a method of identifying candidate non-peptidyl therapeutic agents for modulating smooth muscle (e.g., vascular and/or bronchial) tone comprising: (a) admixing a test agent, a 14-3-3 polypeptide, and a phosphorylated HSP20 polypeptide under conditions that, in the absence of the test agent, would permit interaction of the 14-3-3 and phosphorylated HSP20 polypeptides; (b) determining if the test agent alters the interaction of the 14-3-3 and phosphorylated HSP20 polypeptides; and (c) if the test agent alters the interaction of the 14-3-3 and phosphorylated HSP20 polypeptides, contacting the test agent with smooth muscle (e.g., vascular or bronchial) tissue (in vivo or in vitro) and determining if the test agent alters the contractility and/or tone of the tissue.

Another aspect of the present invention provides a method of identifying a candidate non-peptidyl therapeutic agent for modulating smooth muscle (e.g., vascular and/or bronchial) tone comprising: (a) admixing a test agent, a 14-3-3 polypeptide, such as 14-3-3γ or 14-3-3η, and a cofilin polypeptide under conditions that, in the absence of the test agent, would permit interaction of the 14-3-3 and cofilin polypeptides; (b) determining if the test agent alters the interaction of the 14-3-3 and cofilin polypeptides; and (c) if the test agent alters the interaction of the 14-3-3 and cofilin polypeptides, contacting the test agent with smooth muscle (e.g., vascular or bronchial) tissue (in vivo or in vitro) and determining if the test agent alters the contractility and/or tone of smooth muscle tissue.

In certain embodiments, the test agent of the methods is a small organic molecule. In other embodiments, the test agent of the methods is a carbohydrate or a nucleic acid. In specific embodiments of the methods, effect of the test agent on the interaction of polypeptides is detected in a competitive binding assay. In certain embodiments, the polypeptide or the test agent is labeled with a detectable marker. For example, the detectable marker is selected from the group consisting of: biotin, digoxygenin, green fluorescent protein (GFP), isotopes, polyhistidine, magnetic beads, glutathione S transferase (GST), and fluors such as fluorescein, DTAF, and Bodipy-FL. Optionally, the method of the invention is repeated for a library of different test agents. In preferred embodiments of the methods, the interaction is detected by fluorescence polarization assay, fluorescence resonance energy transfer (FRET) assay, or ELISA.

Examples of small molecule smooth muscle active compounds of the invention that may be used for medical treatments (e.g., asthma and diseases associated with abnormal vasoconstriction) are illustrated below.

As an example, small molecule smooth muscle active compounds of the invention are represented by the general formula I:

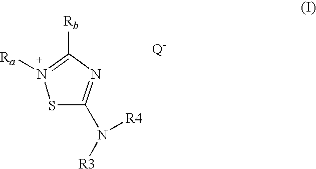

where:

Rₐ is an alkyl, alkenyl, heteroaryl or aryl group;

R_{b} is an alkyl, alkenyl, heteroaryl or aryl group;

R₃ is selected from C1-6 alkyl, arylalkyl, phenyl, heteroaryl, acyl, and sulfonyl;

R₄ is selected from H, C1-6 alkyl, arylalkyl, phenyl and heteroaryl; and

Q⁻ is an anionic counterion, which is preferably suitable for a pharmaceutical preparation.

A preferred group of compounds encompassed by general formula I is represented by general formula II:

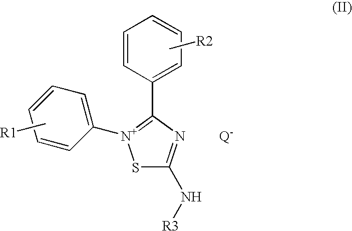

where:

R1 and R2 are independently selected from H, C1-6 alkyl, aryl, halogen, hydroxy, ether, and an optionally substituted amino group;

R3 is selected from C1-6 alkyl, arylalkyl, phenyl, heteroaryl, acyl, and sulfonyl; and

Q⁻ is an anionic counterion, which is preferably suitable for a pharmaceutical preparation.

As another example, small molecule smooth muscle active compounds of the invention are represented by the general formula III:

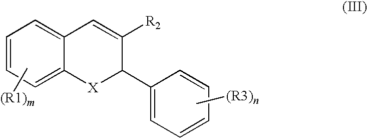

or a pharmaceutically acceptable salt thereof, where:

each R1 and R3 is independently selected from halogen, CF3, C1-6 alkyl, cycloalkyl, amino, hydroxyl, alkoxy, nitro, carboxy, carboxyesters, carboxamide, and sulfonamide, typically each R1 and R3 is independently a halogen, such as bromine or chlorine;

R2 is selected from nitro, carboxy, carboxyester, substituted carboxamide, and C1-6 alkyl;

X is selected from NH and O;

m is an integer from 0 to 4, typically 1 or 2, more typically 2; and

n is an integer from 0 to 5, typically 1 or 2, more typically 2.

As a further example, small molecule smooth muscle active compounds of the invention are represented by the general formula IV:

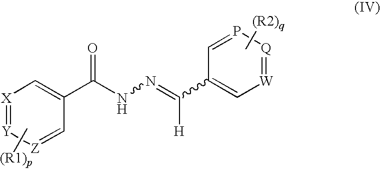

or a pharmaceutically acceptable salt thereof, where:

each R1 and R2 is independently selected from hydroxyl, C1-3 alkoxy, C4-6 cycloalkoxy, nitro, amino, acyl, carboxyl, carboxy ester, carboxamide, and sulfonamide, typically R1 is a halogen such as bromine or chlorine and R2 is hydroxyl or C1-3 alkoxy;

X, Y, Z, P, Q, and W are independently selected from CH and N, typically one of X, Y and Z is N and the remainder are CH and P, Q and W are all CH;

p is an integer from 0 to 5, typically 0 or 1, more typically 0; and

q is an integer from 0 to 5, typically 1 to 3.

### BRIEF DESCRIPTION OF THE DRAWINGS

FIG. 1 shows that once pHSP20 is induced by activation of the smooth muscle cell cyclic nucleotide signaling pathways, it can free pCofilin from its interaction with a 14-3-3 protein (e.g. 14-3-3 gamma or eta), thereby leading to the activation of pCofilin by its dephosphorylation and its subsequent depolymerization of the actin cytoskeleton. Excess unbound pHSP20 is also able to directly destabilize the cytoskeleton. A pHSP20 mimic could substitute for pHSP20 in releasing pCoflin from 14-3-3. The mimic could further act to release pHSP20 itself from 14-3-3, thereby increasing the pool of endogenous free pHSP20 to interact with the cytoskeleton. Finally, a pHSP20 mimic could directly substitute for pHSP20 in its role of destabilizing the cycloskeleton.

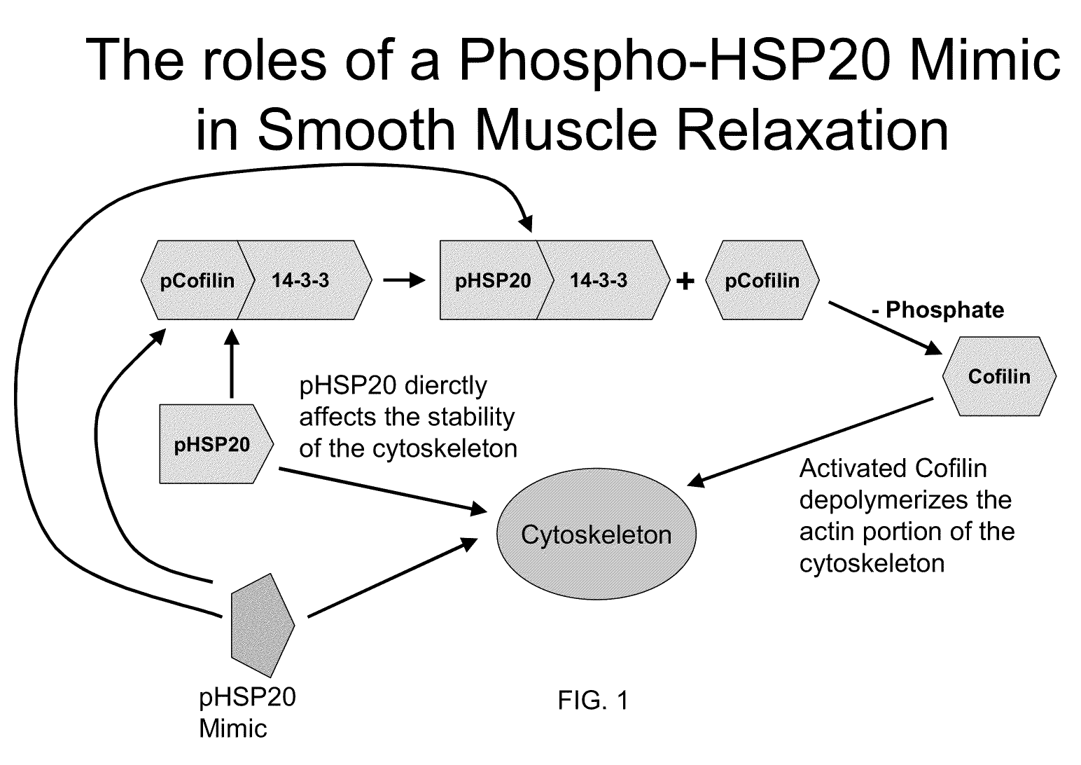

FIG. 2 shows that pHSP20 peptide binds to 14-3-3 proteins. Silver-stained SDS-PAGE analysis is shown for two replicates of pull-down experiments using control ethanolamine beads (lane 1), HSP20 peptide (lanes 2 and 5), pHSP20 peptide (lanes 3 and 6), and scrHSP20 peptide (lanes 4 and 7).

FIG. 3 shows binding of 14-3-3 to the pHSP20 ligand is decreased when free pHSP20 is used as a competitor in a surface plasmon resonance-based (Biacore) experiment. In this competition experiment, the ligand is a derivative of a pHSP20 fragment (WLRRApSAPLPGLK) which is immobilized to a Biacore chip. The competitor pHSP20 is added at various concentrations (0, 340, 680, 1352, 5402 nM). The 14-3-3 protein is the 14-3-3γ isoform (also referred to as YWHAG). In a control experiment, the ligand is non-phosphorylated peptide (HSP20) which is immobilized to a Biacore chip. No binding of 14-3-3 to the ligand is detected.

FIG. 4 shows no detectable competition when non-phosphorylated peptide (HSP20 peptide) is used as a competitor.

FIG. 5 shows strong competition when a minimal 14-3-3 consensus binding sequence (RRApSAP) of pHSP20 is used as a competitor. At each concentration tested, the minimal 14-3-3 binding consensus sequence out competes the original pHSP20 peptide sequence (WLRRApSAPLPGLK) in binding to 14-3-3, as described in Example 2.

FIG. 6 shows strong competition when an alternative 14-3-3 binding sequence (WLRRApSAP) of pHSP20 is used as a competitor. At each concentration tested, the alternative 14-3-3 binding sequence out competes the original pHSP20 peptide sequence (WLRRApSAPLPGLK) in binding to 14-3-3, as described in Example 2

FIG. 7 shows that E25-14-3-3 proteins bind in a Biacore experiment to the immobilized pHSP20 peptide. E25 refers to Biotin-His tagged proteins. 14-3-3γ (YWHAG) and 14-3-3η (YWHAH) bind stronger than the other 14-3-3 isoforms, as described in Example 3.

FIG. 8 shows the kinetics for the interaction between E23-14-3-3γ (also referred to as E23-YWHAG; E23 refers to GST-His tagged proteins) and pHSP20 peptide.

FIG. 9 shows the dose response curves for compounds (a)-(k) in inhibiting the interaction between 14-3-3γ and pHSP20 peptide (WLRRApSAP) in a fluorescence polarization assay, as described in Example 4.

FIG. 10 shows the dose response curves for compound (l), (m) and pHSP20 peptide (WLRRApSAPLPGLK) to inhibit the interaction between 14-3-3γ and pHSP20 in a fluorescence polarization assay, as described in Example 5.

FIG. 11 shows the contraction/dilation of bovine coronary artery rings when exposed to a pHSP20 peptide (PTD-20), a cyclodextrin control (CD), compound (m) and compound (n), as described in Example 6.

FIGS. 12A-C are mean square displacement (MSD) plots for the time control and samples treated with 200 μM sodium arsenite and 1 mM db-cAMP, respectively, as described in Example 7.

FIGS. 13A-D are MSD plots for cells treated with various concentrations of phosphorylated and non-phosphorylated PTD-HSP20 peptide.

FIG. 14 is the MSD plot of cells treated with a 4% cyclodextrin control.

FIGS. 15A-D are MSD plots of non-peptidyl compounds (o), (m), (n) and (f), respectively.

FIG. 16 shows the change of cell stiffness over time for the controls described in Example 8.

FIG. 17 shows the change in cell stiffness caused by compounds of the invention, in comparison to a cyclodextrin control.

### DETAILED DESCRIPTION OF THE INVENTION

### I. Overview

The current invention is based in part on the fact that the phosphorylation of HSP20 plays a key role in the regulation of smooth muscle cell tone and the discovery that compounds that mimic the effect of pHSP20 can be used to affect the tone of smooth muscle tissue. As such, the invention provides treatments for conditions associated with increased or decreased levels of pHSP20.

Additionally, the current invention is based in part on the discovery that interaction of 14-3-3 proteins, such as 14-3-3γ or 14-3-3η, with phosphorylated forms of heat shock protein 20 (pHSP20) plays a role in the regulation of smooth muscle tone. Directing drugs at this interaction, either by promoting or mimicking it or mimicking the effect of pHSP20 itself, or alternatively by inhibiting pHSP20's effect on 14-3-3 proteins (herein “de-repressing pHSP20 inhibition”), can be used to regulate smooth muscle tissue, such as in the regulation of vascular tone, bronchial tone or other smooth-muscle tissues. As such, the invention provides treatments for conditions associated with increased levels of 14-3-3.

The present discovery also provides insight into a likely mechanism by which phosphorylation of HSP20 on serine 16 leads to vasorelaxation. While not wishing to be bound to any particular theory, it is possible that the binding of phosphorylated HSP20 to 14-3-3 proteins prevents those proteins from, in turn, binding and stabilizing phosphorylated cofilin and/or prevents free pHSP20 from being available to affect other aspects of cytoskeletal dynamics. Smooth muscle tone can be influenced by alterations in the dynamic equilibrium between filamentous and monomeric actin. Unphosphorylated cofilin is essential for effective depolymerization of actin filaments, whereas phosphorylation inactivates cofilin, leading to accumulation of actin filaments. By binding to pCofilin, 14-3-3 proteins such as 14-3-3γ or 14-3-3η can maintain the cellular phosphocofilin pool and promote the accumulation of actin filaments and promote smooth muscle constriction (e.g., vasoconstriction). However, phosphorylated HSP20 can compete with cofilin for the binding of 14-3-3 proteins, such as 14-3-3γ or 14-3-3η, and thereby reduce the level of phosphocofilin which in turn promotes actin depolymerization, leading to smooth muscle relaxation (e.g., vasorelaxation or bronchorelaxation). Accordingly, another target for drug intervention provided by the present invention is the cofilin/14-3-3γ and cofilin/14-3-3η interactions.

For ease of reading, the present application refers to HSP20 and cofilin as “14-3-3 ligands.” The term “14-3-3 polypeptide” includes full-length proteins, as well as fragments or mutants which retain the ability to bind to pHSP20 or pCofilin (as appropriate), along with fusion proteins including the full-length protein, fragments or mutants. Likewise, the term “HSP20 polypeptide” refers to full length protein, as well as fragments or mutants thereof which bind to 14-3-3 polypetides, e.g., including the phosphoserine-16 residue. The term “cofilin polypeptide” refers to full-length protein, as well as fragments or mutants thereof which bind to 14-3-3 polypetides, e.g., including phosphoserine-3 residue and/or phosphoserine-23 residues. The term “HSP20/cofilin polypeptide” refers to either an HSP20 polypeptide or a cofilin polypeptide, as appropriate from the context.

As described in more detail below, Applicants have developed screening methods to identify smooth muscle active (sm-active) therapeutic agents that may be useful for modulating smooth muscle tone (e.g., vasorelaxation, vasoconstriction, bronchorelaxation, etc.). In certain embodiments of the present invention, drugs that modulate 14-3-3γ and/or 14-3-3η, such as which mimic or interfere with the 14-3-3 polypeptide/ligand interaction, can be used to alter in vascular smooth muscle cell relaxation, either in vivo or in vitro. Such drugs, depending on whether they agonize or mimic pHSP20's effects on 14-3-3 proteins or derepress pHSP20's inhibitory activity on 14-3-3 proteins, can be used to induce vasoconstriction or vasorelaxafion in an animal or in vascular tissue provided in culture. Both in vivo and in vitro assays are provided that can be used to assess test agents for their ability to modify these interactions and, ultimately, for their effect on vascular tone, airway smooth muscle tone or generally smooth muscle tone in one or more locations.

### II. Further Definitions

As used herein, the term “14-3-3 protein” refers to a member of the 14-3-3 protein family. 14-3-3 is a family of highly homologous proteins encoded by separate genes. There are seven known mammalian 14-3-3 isoforms (Ichimura et al., 1988, PNAS 85:7084-7088; Martin et al., 1993, FEBS Lett. 331:296-303). The 14-3-3 proteins exist mainly as dimers with a monomeric molecular mass of approximately 30 kD. General properties of the 14-3-3 polypeptides can further be found in Fu et al. (2000) Annu. Rev. Pharmacol. Toxicol. 40:617-647; Takahashi, 2003, Neurochem Res. 28:1265-73; and Tzivion and Avruch, 2002, J Biol. Chem. 277:3061-4. The nucleic acid and amino acid sequences of various 14-3-3 family members can be found in, for example, Leffer et al, (1993) J. Mol. Biol. 231:982-998. Homologs of 14-3-3 proteins have also been found in a broad range of eukaryotic organisms.

A preferred 14-3-3 isoform of the present invention is 14-3-3γ. 14-3-3γ has been shown to be expressed in vascular tissues (Autieri and Carbone, 1999, DNA Cell Biol. 18: 555; Autieri, et al., 1996, Cell Growth Differ. 7:1453) and, as described in the appended examples, binds to pHSP20.

The 14-3-3 proteins are thought to be general biochemical regulators because they are involved with many cellular functions and have a broad range of ligands, such as receptors, kinases, phosphatases, and docking molecules. In addition to playing a structural role by stabilizing the activity and conformation of signaling proteins, 14-3-3 proteins also act as scaffolding proteins by interacting with and localizing phosphorlyated motifs (Yaffe et al., 1997, Cell 91: 961).

The heat shock protein 20 (also known in the art as HSP20 or P20) is a member of the heat shock protein superfamily. HSP20 has been shown to be involved in the regulation of vascular tone (Beall, et al., 1999, J Biol. Chem. 274:11344; Beall, et al., 1997, J Biol. Chem. 272:11283; Brophy, et al., 2002, World J. Surg. 26:779; Brophy, et al., 1997, Biol Reprod. 57:1354; Brophy, et al., 1999, J Biol. Chem. 274:6324; Brophy, et al., 1999, J Vasc Surg. 29:326; Fuchs, et al., 2000, Am J Physiol Regul Integr Comp Physiol. 279:R492; Rembold, et al., 2000, J Physiol. 524 Pt 3:865; Tessier, et al., 2003, J Surg Res. 111:152; Macomson, et al., 2002, Neurosurgery. 51:204; Pipkin, et al., 2003, Circulation. 107: 469; Woodrum, et al., 2003, J Vasc Surg. 37:874). Activation of cyclic nucleotide-dependent signaling pathways in relevant tissues leads to phosphorylation of the HSP20 on serine 16, and relaxation of smooth muscle, such as vascular smooth muscle. HSP20 has further been shown to be localized in a variety of vascular tissues. Recently, HSP20 phosphopeptide fragments were shown to induce vasorelaxation (Flynn, et al., 2003, FASEB J. 17:1358).

The term “pharmaceutically active” means any physiologically or pharmacologically active chemical entity that produces a desired local or systemic effect in a treated animal, e.g., in a human patient, and preferably with an ED50 of 1 mM or less, more preferably less than 100 μM and even more preferably less than 10 μM.

A “patient” or “subject” can mean either human or non-human animal.

The term “suitable for use in a human patient” means a pharmaceutically active composition that is below the FDA threshold for pyrogenic contaminants for the intended preparation and route of administration.

A “prodrug” is a compound that may not be pharmacologically active, but is at least less active than a metabolite thereof. That is, the ED₅₀ for a biological activity of a prodrug is usually greater than for one or more of its metabolites. However, when activated in vivo by metabolic (such as enzymatic) or non-enzymatic hydrolytic cleavage, or reductive cleavage (e.g., of a disulfide linkage), the prodrug is converted to a pharmaceutically active moiety. Prodrugs are typically formed by chemical modification of a pharmaceutically active moiety.

The term “ED₅₀” means the dose of a drug which produces 50% of its maximum response or effect. Alternatively, ED₅₀ means the dose which produces a pre-determined response in 50% of test subjects or preparations.

### III. Drug Screening Assays

There are numerous approaches to screening for therapeutic agents for modulating smooth muscle relaxation by targeting the roles of 14-3-3γ and/or 14-3-3η in, for example, vascular tone and bronchial tone. For ease of reading, the discussion below will refer to assays derived to be directed to agents that affect 14-3-3γ. However, one of ordinary skill in the art will readily recognize that similar assays can be derived using 14-3-3η or other 14-3-3 isoforms, as appropriate to find compounds that mimic pHSP20, to modulate 14-3-3 interactions with pHSP20 or pCofilin, to modulate HSP20 phosphorylation in smooth muscle cells, to modulate cofilin dephosphorylation in smooth muscle cells or to generally modulate the cytoskeletal dynamics of smooth muscle cells.

For example, high-throughput screening of compounds can be carried out to identify agents that perturb 14-3-3γ-mediated effects on vasorelaxation, such as which affect pHSP20-mediated effects on 14-3-3γ and/or 14-3-3η-mediated effects on cofilin. In certain embodiments, the assay is carried out to screen and identify compounds that specifically inhibit or reduce binding of 14-3-3γ to its binding partner (e.g., pHSP20 or pCofilin). Alternatively, the assay can be used to identify compounds that enhance binding of 14-3-3γ to its binding protein (e.g., pHSP20 or pCofilin). Compounds identified through this screening can be tested in vascular tissues to assess their ability to modulate smooth muscle relaxation (e.g., vasorelaxation or bronchorelaxation) in vitro. Optionally, these compounds can further be tested in animal models to assess their ability to modulate vascular tone in vivo.

A variety of assay formats will suffice and, in light of the present disclosure, those not expressly described herein will nevertheless be comprehended by one of ordinary skill in the art. Agents to be tested for their ability to act as modulators of 14-3-3γ-mediated smooth muscle tone can be produced, for example, by bacteria, yeast, plants or other organisms (e.g., natural products), produced chemically (e.g., small molecules, including peptidomimetics), or produced recombinantly. Test agents contemplated by the present invention include non-peptidyl organic molecules, sugars, hormones, and nucleic acid molecules (such as antisense or RNAi nucleic acid molecules). In a preferred embodiment, the test agent is a small organic molecule having a molecular weight of less than about 2,000 daltons.

The test agents can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity between 14-3-3γ and other proteins, or in changes in a property of the molecular target for 14-3-3γ binding (such as regulation of cofilin phosphorylation or the amount of free (i.e., unbound) pHSP20).

Merely to illustrate, in an exemplary screening assay of the present invention, the compound of interest is contacted with an isolated and purified 14-3-3γ polypeptide which is ordinarily capable of binding pHSP20 or pCofilin polypeptides, as appropriate for the intention of the assay. To the mixture of the compound and 14-3-3γ polypeptide is then added a composition containing a pHSP20 or pCofilin polypeptide. Detection and quantification of 14-3-3γ complexes provides a means for determining the compound's efficacy at inhibiting (or potentiating) complex formation between the 14-3-3γ and pHSP20/pCofilin polypeptides. The efficacy of the compound can be assessed by generating dose response curves from data obtained using various concentrations of the test compound. Moreover, a control assay can also be performed to provide a baseline for comparison. In the control assay, isolated and purified pHSP20 or pCofilin is added to a composition containing the 14-3-3γ polypeptide, and the formation of 14-3-3γ complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between the 14-3-3γ polypeptide and target polypeptide may be detected by a variety of techniques. For instance, modulation of the formation of complexes can be quantitated using, for example, detectably labeled proteins such as radiolabelled (e.g., 32P, 35S, 14C or 3H), fluorescently labeled (e.g., FITC), or enzymatically labelled 14-3-3γ or pHSP20/pCofilin polypeptides, by immunoassay, or by chromatographic detection.

In certain embodiments, it will be desirable to immobilize either the 14-3-3γ or the pHSP20/pCofilin polypeptide to facilitate separation of protein complexes from uncomplexed forms of one or both of the proteins, as well as to accommodate automation of the assay. Binding of the pHSP20/pCofilin polypeptide to 14-3-3γ, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-5-transferase/14-3-3γ (GST/14-3-3γ) fusion proteins can be adsorbed onto glutathione sepharose beads (Sigma Chemical, St. Louis, Mo.) or glutathione derivatized microtitre plates, which are then combined with the pHSP20/pCofilin polypeptide, e.g., an 35S-labeled pHSP20/pCofilin polypeptide, and the test compound, and the mixture incubated under conditions conducive to complex formation, e.g., at physiological conditions for salt and pH, though slightly more stringent conditions may be desired. Following incubation, the beads are washed to remove any unbound pHSP20/pCofilin polypeptide, and the matrix immobilized radiolabel determined directly (e.g., beads placed in scintilant), or in the supernatant after the protein complexes are subsequently dissociated. Alternatively, the complexes can be dissociated from the matrix, separated by SDS-PAGE, and the level of pHSP20/pCofilin polypeptide found in the bead fraction quantitated from the gel using standard electrophoretic techniques.

Other techniques for immobilizing proteins on matrices are also available for use in the subject assay. For instance, either of the 14-3-3γ or pHSP20/pCofilin polypeptides can be immobilized utilizing conjugation of biotin and streptavidin. For instance, biotinylated 14-3-3γ molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the 14-3-3γ but which do not interfere with pHSP20/pCofilin binding can be derivatized to the wells of the plate, and the 14-3-3γ trapped in the wells by antibody conjugation. As above, preparations of a pHSP20/pCofilin polypeptide and a test compound are incubated in the 14-3-3γ-presenting wells of the plate, and the amount of protein complex trapped in the well can be quantitated. Exemplary methods for detecting such complexes, in addition to those described above for the GST-immobilized complexes, include immunodetection of complexes using antibodies reactive with the pHSP20/pCofilin polypeptide, or which are reactive with the 14-3-3γ protein and compete for binding with the pHSP20/pCofilin polypeptide; as well as enzyme-linked assays which rely on detecting an enzymatic activity associated with the pHSP20/pCofilin polypeptide. In the instance of the latter, the enzyme can be chemically conjugated or provided as a fusion protein with a pHSP20/pCofilin polypeptide. To illustrate, the pHSP20/pCofilin polypeptide can be chemically cross-linked or genetically fused with horseradish peroxidase, and the amount of pHSP20/pCofilin polypeptide trapped in the complex can be assessed with a chromogenic substrate of the enzyme, e.g., 3,3′-diamino-benzadine terahydrochloride or 4-chloro-1-napthol. Likewise, a fusion protein comprising the pHSP20/pCofilin polypeptide and glutathione-5-transferase can be provided, and complex formation quantitated by detecting the GST activity using 1-chloro-2,4-dinitrobenzene (Habig et al (1974) J Biol Chem 249:7130).

In certain embodiments, the assay is carried out to screen and identify compounds that specifically inhibit or reduce binding of 14-3-3γ to its binding partner (e.g., pHSP20 or pCofilin), by inhibition of binding of a labeled 14-3-3 protein or fragments thereof to an immobilized 14-3-3 binding protein (e.g., pHSP20 or pCofilin). Alternatively, such libraries can be similarly screened to identify members which enhance binding of 14-3-3 to its binding protein (e.g., pHSP20 or pCofilin). Compounds identified through this screening can be tested in vascular tissues to assess their ability to modulate vasorelaxation in vitro. Optionally, these compounds can further be tested in animal models to assess their ability to modulate vascular tone or other smooth muscle tone in vivo.

In another embodiment, fluorescence polarization assays are used in the methods of the invention. To illustrate, a 14-3-3 ligand (e.g., pHSP20 peptide or pCofilin peptide) is conjugated to a small molecule fluorophore such as fluorescein or Oregon green. Binding of the tagged 14-3-3 ligand to a purified 14-3-3 polypeptide would cause a decrease in the mobility of the 14-3-3 ligand and thus, increase the polarization of the emitted light from the fluorophore. This technique thereby allows for measuring, either directly or indirectly, the degree of interaction between a 14-3-3 protein and a 14-3-3 ligand (e.g., pHSP20 peptide or pCofilin peptide) in the presence or absence of a test agent. Accordingly, agents that modulate (increase or decrease) the 14-3-3/ligand interaction can be identified.

In another specific embodiment, fluorescence resonance energy transfer (FRET) assays are used in the methods of the invention. These assays utilize two fluorescently tagged species, where the emission spectrum of the shorter wavelength tag overlaps the excitation spectrum of the longer wavelength tag. Close proximity of the two molecules induced by binding allows nonradiative excitation of the long wavelength tag when the short wavelength tag is excited. To illustrate, two DNA expression constructs coding for the 14-3-3 polypeptide and the 14-3-3 ligand respectively are tagged with ECFP(cyan) and EYFP(yellow). Upon expression in vivo, energy transfer in the cell lysates can be observed. It is recognized that such assays can be adapted to an in vitro format. This technique thereby allows for measuring, either directly or indirectly, the degree of interaction between a 14-3-3 protein and a 14-3-3 ligand (e.g., pHSP20 or pCofilin) in the presence or absence of a test agent. Accordingly, agents that modulate (increase or decrease) the 14-3-3/ligand interaction can be identified.

Furthermore, other modes of detection such as those based on optical waveguides (PCT Publication WO 96/26432 and U.S. Pat. No. 5,677,196), surface plasmon resonance (SPR), surface charge sensors, and surface force sensors are compatible with many embodiments of the invention.

Moreover, the subject polypeptides can be used to generate an interaction trap assay, also known as the “two-hybrid assay,” for identifying agents that disrupt or potentiate binding of 14-3-3γ to a pHSP20 or pCofilin. See for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present invention contemplates the use of reverse two-hybrid systems to identify compounds (e.g., small molecules or peptides) that dissociate interactions between 14-3-3γ and its ligand (e.g., pHSP20 or pCofilin). See for example, Vidal and Legrain, (1999) Nucleic Acids Res 27:919-29; Vidal and Legrain, (1999) Trends Biotechnol 17:374-81; and U.S. Pat. Nos. 5,525,490; 5,955,280; 5,965,368.

The interaction trap assay relies on reconstituting a functional transcriptional activator protein from two separate fusion proteins, one of which comprises the DNA-binding domain of a transcriptional activator fused to a 14-3-3γ polypeptide. The second fusion protein comprises a transcriptional activation domain (e.g., able to initiate RNA polymerase transcription) fused to a pHSP20 or pCofilin polypeptide. When the 14-3-3γ and pHSP20/pCofilin domains of each fusion protein interact, the two domains of the transcriptional activator protein are brought into sufficient proximity as to cause transcription of a reporter gene. By detecting the level of transcription of the reporter, the ability of a test agent to inhibit (or potentiate) binding of 14-3-3γ to pHSP20 or pCofilin can be evaluated.

In an illustrative embodiment, Saccharomyces cerevisiae YPB2 cells are transformed simultaneously with a plasmid encoding a GAL4bd-14-3-3γ fusion and with a plasmid encoding the GAL4ad domain fused to a pHSP20 or pCofilin. Moreover, the strain is transformed such that the GAL4-responsive promoter drives expression of a phenotypic marker. For example, the ability to grow in the absence of histidine can depend on the expression of the HIS3 gene. When the HIS3 gene is placed under the control of a GAL4-responsive promoter, relief of this auxotrophic phenotype indicates that a functional GAL4 activator has been reconstituted through the interaction of 14-3-3γ and the pHSP20 or pCofilin. Thus, a test agent able to inhibit this interaction with 14-3-3γ will result in yeast cells unable to grow in the absence of histidine. Alternatively, the phenotypic marker (e.g., instead of the HIS3 gene) can be one which provides a negative selection (e.g., are cytotoxic) when expressed such that agents which disrupt 14-3-3γ interactions confer positive growth selection to the cells. Yeast cells bearing other interaction pairs can be used to evaluate the specificity of a given protein-protein interaction inhibitor.

After identifying an agent using a cell-free system, or any other agent that is expected to effect 14-3-3γ-mediated activity, the subject test agents can be tested in whole cells or tissues, in vitro or in vivo, to confirm their ability to modulate vascular tone. Various methods known in the art can be utilized to test the vasorelaxing or vascular constricting activity of a candidate agent. See, for example, Tessier et al., 2003, J Surg Res. 111:152-7; Woodrum et al., 2003, J Vasc Surg. 37:874-81; and Brophy et al., 2002, J Vasc Res. 39:95-103.

In a specific embodiment, methods of the invention are carried out in intact strips of vascular smooth muscle. Transverse strips of bovine carotid artery smooth muscle, denuded of endothelium, are suspended in a muscle bath containing bicarbonate buffer (120 mM NaCl, 4.7 mM KCl, 1.0 mM MgSO₄, 1.0 mM NaH₂PO₄, 10 mM glucose, 1.5 mM CaCl₂, and 25 mM Na₂HCO₃, pH 7.4), equilibrated with 95% O2/5% CO2, at 37° C. at one gram of tension for 2 hours. The muscles are pre-contracted with serotonin (1 μM for 10 minutes) and cumulative doses of test agents are added. The force is depicted as a percentage of the maximal serotonin contraction (n=5, *=p<0.05 compared to no test agent added). If a test agent decreases the contractile force in serotonin pre-contracted artery smooth muscles, then the test agent is able to relax and prevent spasm in vascular smooth muscles. Alternatively, if a test agent increases the contractile force in serotonin pre-contracted artery smooth muscles, then the test agent is able to constrict and prevent relaxation in vascular smooth muscles. It will be recognized by those of skill in the art that this method may be applied to other types of smooth muscle tissue, for example airway smooth muscle.

In another specific embodiment, methods of the invention are carried out in cultured rat aortic smooth muscle cells. Contractile function is monitored using the silicone polymer wrinkle assay to determine contractility in cultured mesangial cells. In the presence of serum, cells form wrinkles on the polymer, indicating of contraction. If a test agent reduces wrinkling on the polymer in response to serum, then the test agent is able to relax and prevent spasm in smooth muscles. Alternatively, if a test agent increases wrinkling on the polymer in response to serum, then the test agent is able to constrict and prevent relaxation in vascular smooth muscles. It will be recognized by those of skill in the art that this method may be applied to other types of smooth muscle tissue, for example airway smooth muscle.

In a further embodiment, the present invention contemplates methods of optimizing the structure of a candidate therapeutic compound once the candidate therapeutic compound is identified by the methods as described above. Preferably, the candidate therapeutic compound is a small molecule, and it modulates the 14-3-3/ligand interaction by binding to the 14-3-3 protein or binding to pHSP20 or pCofilin. For example, the structure of the identified small molecule may be optimized to increase its efficiency in modulating the vasoactive properties of HSP20 by using the information obtained from a co-crystal structure of a vasoactive fragment of pHSP20 and its target 14-3-3 protein.

In other embodiments, other assays can be used to screen for compounds that decrease the expression level (protein or nucleic acid) of 14-3-3γ protein or HSP20 or cofilin or alternatively increase the expression level (protein or nucleic acid) of 14-3-3γ protein or HSP20 or cofilin. Methods of detecting and optionally quantitating proteins can be achieved by techniques such as antibody-based detection assays. In these cases, antibodies may be used in a variety of detection techniques, including enzyme-linked immunosorbent assays (ELISAs), immunoprecipitations, and Western blots. On the other hand, methods of detecting and optionally quantitating nucleic acids generally involve preparing purified nucleic acids and subjecting the nucleic acids to a direct detection assay or an amplification process followed by a detection assay. Amplification may be achieved, for example, by polymerase chain reaction (PCR), reverse transcriptase (RT), and coupled RT-PCR. Detection of nucleic acids is generally accomplished by probing the purified nucleic acids with a probe that hybridizes to the nucleic acids of interest, and in many instances, detection involves an amplification as well. Northern blots, dot blots, microarrays, quantitative PCR, and quantitative RT-PCR are all well known methods for detecting nucleic acids.

In some cases, one or more compounds can be tested simultaneously. Where a mixture of compounds is tested, the compounds selected by the foregoing processes can be separated (as appropriate) and identified by suitable methods (e.g., PCR, sequencing, chromatography). Large combinatorial libraries of compounds (e.g., organic compounds, peptides, nucleic acids) produced by combinatorial chemical synthesis or other methods can be tested (see e.g., Ohlmeyer, M. H. J. et al., Proc. Natl. Acad. Sci. USA 90:10922-10926 (1993) and DeWitt, S. H. et al., Proc. Natl. Acad. Sci. USA 90:6909-6913 (1993), relating to tagged compounds; see also, Rutter, W. J. et al., U.S. Pat. No. 5,010,175; Huebner, V. D. et al., U.S. Pat. No. 5,182,366; and Geysen, H. M., U.S. Pat. No. 4,833,092). Where compounds selected from a combinatorial library by the present method carry unique tags, identification of individual compounds by chromatographic methods is possible. Where compounds do not carry tags, chromatographic separation, followed by mass spectrophotometry to ascertain structure, can be used to identify individual compounds selected by the method, for example.

### IV. Compositions and Smooth Muscle Active Agents of the Invention

Agents identified to have effect on 14-3-3γ-mediated, 14-3-37′-mediated and/or (p)HSP20 smooth muscle cell activity (collectively herein “smooth muscle active agents” or “sm-active agents”, including vasoactive and bronchoactive agents), such as by the assays described above, can be used to generate compositions, e.g., suitable for use in human patients, that modulate vascular tone, bronchial tone or other smooth muscle tissues. For example, vasoactive agents can enhance vasodilation, enhance vasoconstriction, or increase blood flow. Bronchoactive agents can, as appropriate, enhance bronchodilation or enhance bronchoconstriction. In certain cases, these agents are capable of relaxing or constricting vascular, bronchial or smooth muscle.

In certain embodiments, the sm-active agent is a small organic molecule, e.g., has a molecular weight less than 2000 amu, and even more preferably less than 1500 or even 1000 amu. Preferably the agent is cell-permeable. In certain preferred embodiments, the agent is also orally active. Candidate agents comprise functional groups necessary for structural interaction with proteins, particularly hydrogen bonding, and typically include at least an amine, carbonyl, hydroxyl, sulfhydryl or carboxyl group. Candidate small molecule compounds can be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant, and animal extracts are available or readily produced. Additionally, natural or synthetically produced libraries and compounds can be modified through conventional chemical, physical, and biochemical means. Known pharmacological agents may be subjected to directed or random chemical modifications, such as acylation, alkylation, esterification, and amidification, to produce structural analogs.

As an example, small molecule vasoactive compounds of the invention are represented by the general formula I:

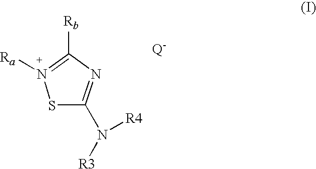

where:

Rₐ is an alkyl, alkenyl, heteroaryl or aryl group;

R_{b} is an alkyl, alkenyl, heteroaryl or aryl group;

R₃ is selected from C1-6 alkyl, arylalkyl, phenyl, heteroaryl, acyl, and sulfonyl;

R₄ is selected from H, C1-6 alkyl, arylalkyl, phenyl and heteroaryl; and

Q⁻ is an anionic counterion, which is preferably suitable for a pharmaceutical preparation.

In one embodiment, Rₐ is an alkenyl group.

In one embodiment, R_{b} is an alkyl group. In a specific embodiment, R_{b} is an alkyl group and Rₐ is an alkenyl group.

In another embodiment, Rₐ is an alkyl group. In a specific embodiment, Rₐ is an alkyl group and R_{b} is an aryl group. Suitable alkyl groups include phenylalkyl and phenylsulfonylalkyl groups.

In a preferred embodiment, Rₐ is an aryl group, preferably a phenyl group. In a particularly preferred embodiment, Rₐ is an aryl group, preferably a phenyl group, and R_{b} is an aryl group, preferably a phenyl group.

A particularly preferred group of compounds encompassed by this embodiment is represented by general formula II:

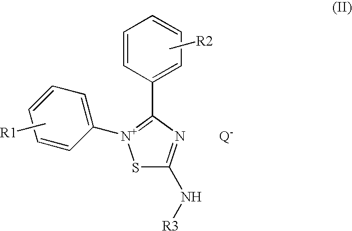

where:

R1 and R2 are independently selected from H, C1-6 alkyl, aryl, halogen, hydroxy, ether, and an optionally substituted amino group;

R3 is selected from C1-6 alkyl, arylalkyl, phenyl, heteroaryl, acyl, and sulfonyl; and

Q⁻ is an anionic counterion, which is preferably suitable for a pharmaceutical preparation.

In one embodiment, R1 and R2 are each hydrogen.

Examples of Q⁻ include chloride, bromide, perchlorate, oxalate, mesylate and sulfate. Typically, Q⁻ is chloride or bromide.

Specific compounds encompassed by general formula I are represented by the following formulae:

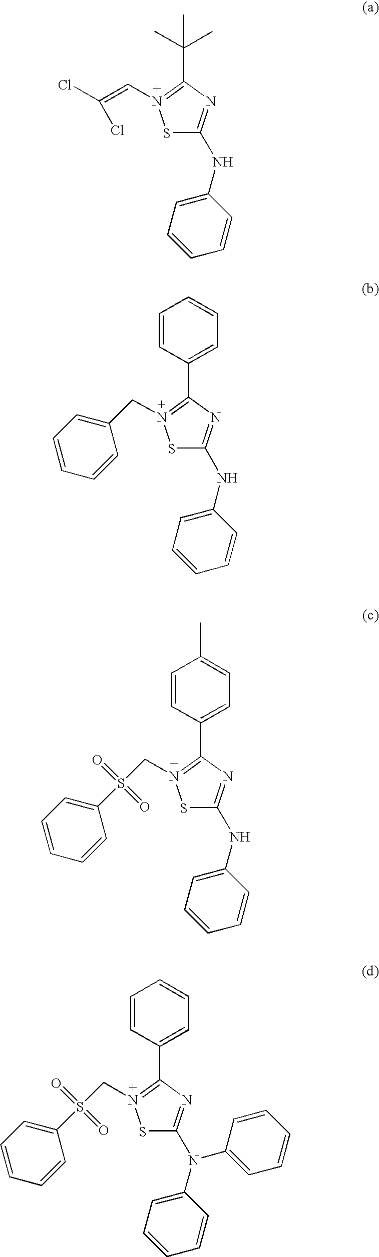

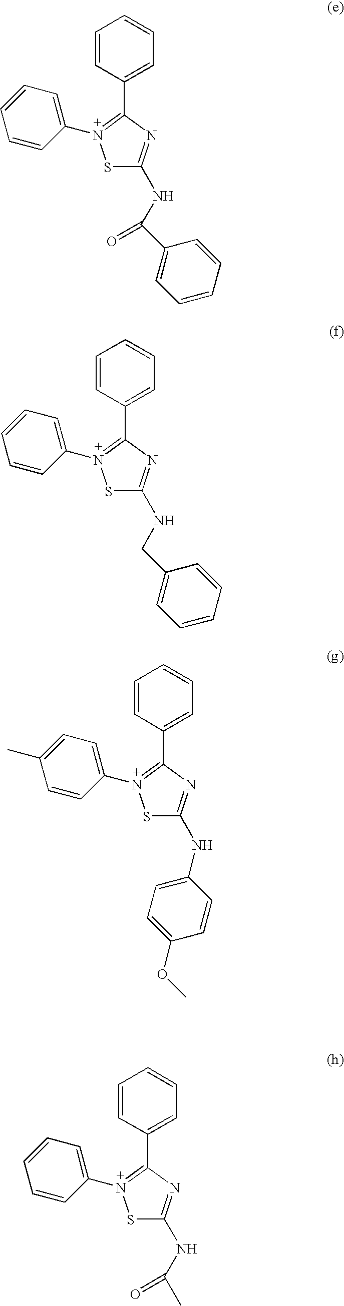

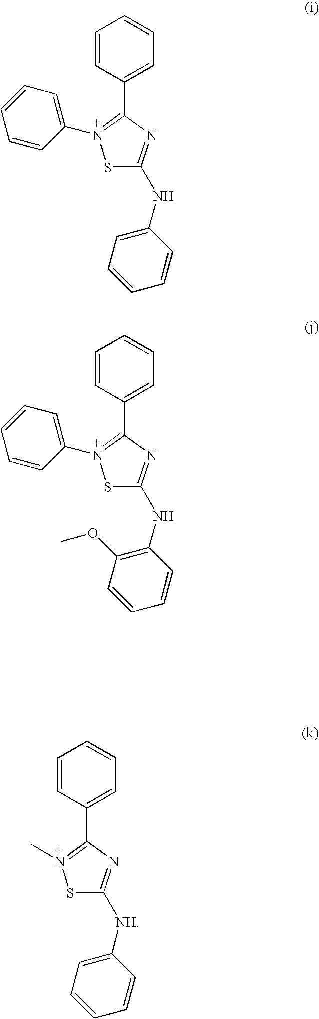

The counterions for compounds (a)-(k) are Q⁻, as defined above.

As another example, small molecule vasoactive compounds of the invention are represented by the general formula III:

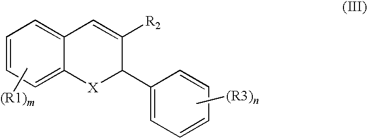

or a pharmaceutically acceptable salt thereof, where:

each R1 and R3 is independently selected from halogen, CF3, C1-6 alkyl, cycloalkyl, amino, hydroxyl, alkoxy, nitro, carboxy, carboxyesters, carboxamide and sulfonamide, typically each R1 and R3 is independently a halogen, such as bromine or chlorine;

R2 is selected from nitro, carboxy, carboxyester, substituted carboxamide, and C1-6 alkyl;

X is selected from NH and O;

m is an integer from 0 to 4, typically 1 or 2, more typically 2; and

n is an integer from 0 to 5, typically 1 or 2, more typically 2.

One compound encompassed by general formula III is represented by the following structural formula:

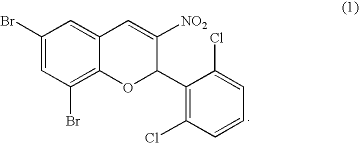

As a further example, small molecule vasoactive compounds of the invention are represented by the general formula IV:

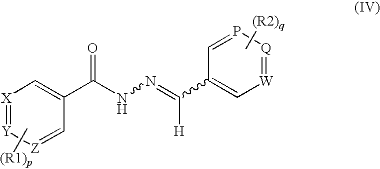

or a pharmaceutically acceptable salt thereof, where:

each R1 and R2 is independently selected from hydroxyl, C1-3 alkoxy, C4-6 cycloalkoxy, nitro, amino, acyl, carboxyl, carboxy ester, carboxamide, and sulfonamide, typically R1 is a halogen such as bromine or chlorine and/or R2 is hydroxyl or C1-3 alkoxy;

X, Y, Z, P, Q, and W are independently selected from CH and N, typically one of X, Y and Z is N and the remainder are CH and P, Q and W are all CH;

p is an integer from 0 to 5, typically 0 or 1, more typically 0; and

q is an integer from 0 to 5, typically 1 to 3.

The conformation about the imine bond can be either cis or trans, but is preferably trans.

Compounds encompassed by general formula IV are represented by the following structural formulae:

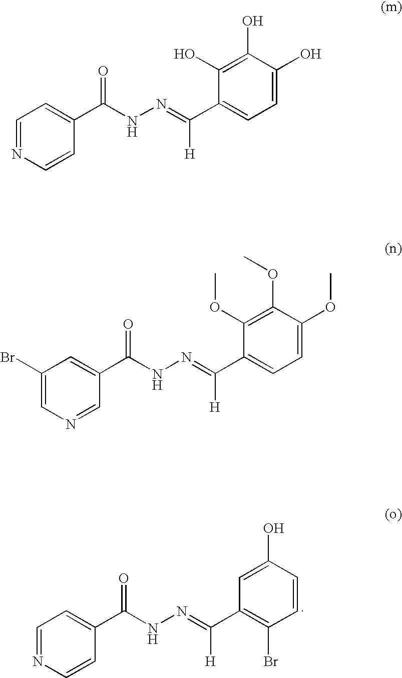

It is contemplated that all embodiments of the invention can be combined with one or more other embodiments, even those described under different aspects of the invention.

The term “acyl” as used herein includes such moieties as can be represented by the general formula:

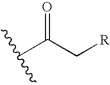

wherein suitable R groups, include, but are not limited to H, alkyl, alkoxy, aralkyl, aryloxy, aryl, heteroaryl, heteroaralkyl, heteroaryloxy, and cycloalkyl, wherein any of these groups may optionally be further appropriately substituted.

The term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. Co alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkoxy” refers to an oxygen having an alkyl group attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “aralkyl”, as used herein, refers to an alkyl group substituted with an aryl group.

The term “carbocyclic” as used herein includes 3- to 8-membered substituted or unsubstituted single-ring saturated or unsaturated cyclic aliphatic groups in which each atom of the ring is carbon.

The term “heterocyclic” as used herein includes 3- to 8-membered, preferably 4- to 8-membered, substituted or unsubstituted single-ring cyclic groups in which the ring includes 1 to 3 heteroatoms.

The term “aryl” as used herein includes 5-, 6-, and 7-membered substituted or unsubstituted single-ring aromatic groups in which each atom of the ring is carbon. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The terms “heteroaryl” includes substituted or unsubstituted aromatic 5- to 7-membered ring structures, more preferably 5- to 6-membered rings, whose ring structures include one to four heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, triazole, pyrazole, pyridine, pyrazine, pyridazine and pyrimidine, and the like.

The term “heteroatom” as used herein means an atom of any element other than carbon or hydrogen. Preferred heteroatoms are nitrogen, oxygen, phosphorus, and sulfur.

The terms “polycyclyl” or “polycyclic” refer to two or more rings (e.g., cycloalkyls, cycloalkenyls, cycloalkynyls, aryls, heteroaryls, and/or heterocyclyls) in which two or more carbons are common to two adjoining rings, e.g., the rings are “fused rings”. Each of the rings of the polycycle can be substituted or unsubstituted.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “non-peptidyl” refers to compounds having no more than two alpha-amino acids connected by an amide linkage. Compounds having three or more alpha-amino acids connected in series by amide linkages are “peptidyl” for purposes of this invention.

In certain other embodiments, the vasoactive agents of the present invention include antisense nucleic acids. In one embodiment, the invention relates to the use of antisense nucleic acids which inhibit expression of HSP20, 14-3-3γ, 14-3-3η, or cofilin polypeptides or variants thereof, to decrease expression of one or more of these polypeptides. Such antisense nucleic acids can be delivered, for example, as an expression plasmid which, when transcribed in the cell, produces RNA which is complementary to at least a unique portion of the cellular mRNA which encodes an HSP20, 14-3-3γ, 14-3-3η, or cofilin polypeptides. Alternatively, the construct is an oligonucleotide which is generated ex vivo and which, when introduced into the cell causes inhibition of expression by hybridizing with the mRNA and/or genomic sequences encoding an HSP20, 14-3-3γ, or cofilin polypeptide. Such oligonucleotide probes are optionally modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in nucleic acid therapy have been reviewed, for example, by van der Krol et al., 1988, Biotechniques 6:958-976; and Stein et al., 1988, Cancer Res 48:2659-2668.

In another embodiment, the invention relates to the use of RNA interference (RNAi) to reduce expression of HSP20, 14-3-3γ, 14-3-3η, or cofilin. RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. RNAi provides a useful method of inhibiting gene expression in vitro or in vivo. RNAi constructs can comprise either long stretches of dsRNA identical or substantially identical to the target nucleic acid sequence or short stretches of dsRNA identical to or substantially identical to only a region of the target nucleic acid sequence.

As used herein, the term “RNAi construct” is a generic term including small interfering RNAs (siRNAs), hairpin RNAs, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

Optionally, the RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

The subject RNAi constructs can be “small interfering RNAs” or “siRNAs.” These nucleic acids are around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group. In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from a Drosophila embryo, thereby producing a combination. The combination is maintained under conditions in which the dsRNA is processed to RNA molecules of about 21 to about 23 nucleotides. The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with an antibody can be used to purify siRNAs.

Alternatively, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In another embodiment, the invention relates to the use of ribozyme molecules designed to catalytically cleave an mRNA transcripts to prevent translation of mRNA (see, e.g., PCT International Publication WO90/11364, published Oct. 4, 1990; Sarver et al., 1990, Science 247:1222-1225; and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art and is described more fully in Haseloff and Gerlach, 1988, Nature, 334:585-591. The ribozymes of the present invention also include RNA endoribonucleases (hereinafter “Cech-type ribozymes”) such as the one which occurs naturally in Tetrahymena thermophila (known as the IVS or L-19 IVS RNA) and which has been extensively described (see, e.g., Zaug, et al., 1984, Science, 224:574-578; Zaug and Cech, 1986, Science, 231:470-475; Zaug, et al., 1986, Nature, 324:429-433; published International patent application No. WO88/04300 by University Patents Inc.; Been and Cech, 1986, Cell, 47:207-216).

In a further embodiment, the invention relates to the use of DNA enzymes to inhibit expression of HSP20, 14-3-3γ, 14-3-3η, or cofilin genes. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid. Briefly, to design an ideal DNA enzyme that specifically recognizes and cleaves a target nucleic acid, one of skill in the art must first identify the unique target sequence. Preferably, the unique or substantially unique sequence is a G/C rich region of approximately 18 to 22 nucleotides. High G/C content helps insure a stronger interaction between the DNA enzyme and the target sequence. When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462.

### V. Methods of Treatment

Sm-active compounds may be used to treat or prevent pathophysiologic conditions which result from, or involve, lack of or undesired constriction of smooth muscle, or those which necessitate therapeutic intervention to achieve or inhibit smooth muscle relaxation.

One embodiment of the invention relates to the administration of a therapeutically effective mount of a sm-active compound to an animal to relax airway smooth muscle. The term “airway smooth muscle” refers to the smooth muscle lining the bronchi or tracheal region. As a result, these compounds may be administered as therapeutic agents for the treatment or prevention of respiratory disorders. The term “respiratory disorder” refers to any impairment of lung function which involves constriction of airways and changes in blood gas levels or lung function. For example, airway obstruction constitutes a respiratory disorder which occurs as a result of acute pulmonary impairment or obstructive lung disease. Severe airway obstruction may ultimately result in life-threatening respiratory failure. Airway obstruction occurs in patients with chronic obstructive lung diseases, such as emphysema and bronchitis. These patients often experience recurrent episodes of respiratory failure as a result of severe airway obstruction. Emphysema can result in significant disability due to dyspnea, extreme restriction of physical activity, and mortality.

Airway obstruction also results from asthma, a disorder characterized by increased responsiveness of the tracheobronchial tree to various stimuli, and which leads to generalized airway constriction manifested by dyspnea, cough and wheezing. Asthma sufferers often experience acute exacerbations of bronchoconstriction, which may be life-threatening.

Another obstructive lung disease, cystic fibrosis, results from abnormal exocrine gland function. Clinical manifestations include excessive mucous secretion, hypertrophy of bronchial glands, infection, and inflammatory and structural changes in the airways which lead to obstruction and ventilation-perfusion imbalance.

Acute respiratory failure my result not only from obstructive disease, but also as a consequence of airway constriction secondary to pneumonia, thromboembolism, left ventricular failure and pneumothorax. Acute respiratory failure may also result from ventilation-perfusion imbalance.

In addition to the treatment or prevention of respiratory disorders, sm-active compounds may also be used to facilitate diagnostic and therapeutic bronchoscopy. The term “bronchoscopy” refers to the procedure in which a flexible fiberoptic, or rigid bronchoscope is introduced into the tracheobronchial tree for the purpose of bronchial visualization, lung biopsy or brushings, aspiration of secretions, and delivery of pharmacological agents.

A complication of bronchoscopy, and thus an impediment to the successful completion of the procedure, is bronchospasm. Patients with a prior history of bronchospasm are particularly at risk for acute enhancement of spasm. Thus, sm-active compounds may also be used to relax airway smooth muscle and eliminate bronchoscopy-induced bronchospasm.

Another embodiment of the invention relates to the administration of a therapeutically effective mount of a sm-active compound to an animal to relax gastrointestinal smooth muscle. The term “gastrointestinal smooth muscle” refers to smooth muscle which is contained in all areas of the gastrointestinal tract. Such areas include, but are not limited to, the esophagus, duodenum, sphincter of Oddi, biliary tract, ileum, sigmoid colon, pancreatic duct and common bile duct. Sm-active compounds may be used for the treatment or prevention of gastrointestinal disorders. Disorders of the gastrointestinal tract include achalasia (spasm of the lower esophageal sphincter), diarrhea, dumping syndrome, and irritable bowel.

An additional embodiment of the invention relates to the administration of sm-active compounds to alleviate contraction or spasm of gastrointestinal smooth muscle, and thus facilitate successful completion of endoscopic procedures. Contraction or spasm of gastrointestinal smooth muscle imposes a technical obstacle which must frequently be overcome in order to enable the clinician to successfully, perform endoscopic procedures.

The term “endoscopic procedures” refers to those diagnostic procedures which utilize an instrument which is introduced into the gastrointestinal tract to provide direct visualization of the gastrointestinal tract, for examination and therapeutic purposes. Such purposes include direct visualization, biopsy, access to the common bile duct, fluid aspiration and removal of foreign bodies, polyps, and other lesions. An example of a particular endoscopic procedure is esophagogastro-duodenoscopy, which is utilized for examination of the esophageal lumen, stomach and duodenum. Another example, endoscopicretrograde cholangiopanereatography (ERCP), enables visualization of the pancreatic duct, common bile duct and the entire biliary tract, including the gall bladder. Further examples of endoscopic procedures are colonoscopy and sigmoidoscopy.

Another embodiment of the invention relates to administration of a therapeutically effective mount of an sm-active compound to relax corpus cavernosum smooth muscle. The term “corpus cavernosum” refers to two areas of smooth muscle which lie side by side on the dorsal aspect of the penis, and together with the corpus spongeosum that surrounds the urethra, constitute erectile tissue. This erectile tissue consists of an irregular sponge-like system of vascular spaces interspersed between arteries and veins. Erection occurs when cavernosa smooth muscle relaxation causes a decrease in arterial resistance and resulting increase in arterial blood flow to the penis.

Smooth muscle has a critical role in erectile function. Thus, another embodiment of the invention relates to the administration of a therapeutically effective mount of a sm-active compound for the treatment of impotence. “Impotence” refers to a condition of male sexual dysfunction which is characterized by the inability to obtain or maintain an erection.

Organic causes of erectile impotence may include endocrine, drug-induced, local injury, neurologic, and vascular. In particular, impotence may result from neurologic blockade caused by such drugs as antihistamines, antihypertensives, psychogenic agents, and anticholinergics. Impotence may also result from neurologic disorders such as interior temporal lobe lesions, spinal cord disorders, and insufficiency of sensory input resulting from diabetic neuropathy. An additional cause of impotence is insufficient blood flow into the vascular network resulting from an intrinsic defect, or from penile trauma.

Another embodiment of the claimed invention relates to the administration of a therapeutically effective amount of a sm-active compound to relax bladder smooth muscle. Bladder smooth muscle includes that of the bladder base, bladder body and proximal urethra. In addition, sm-active compounds may be used for the treatment of bladder dysfunction disorders, which involve relaxation of bladder smooth muscle. Such disorders include, but are not limited to, problems with bladder filling, volume and continence.

In addition, sm-active compounds may be administered to cause relaxation of urethral and bladder base smooth muscle, and thus, facilitate cystoscopic examination of the urinary tract. The term “cystoscopic examination” refers to the introduction of a fiberoptic instrument through the urethra and into the bladder, to achieve visualization of the interior of the urethra and bladder for diagnostic and therapeutic purposes.

Another embodiment of the invention relates to the administration of a therapeutically effective amount of a sm-active compound to relax uterine smooth muscle. Increased contractility of uterine smooth muscle precipitates premature labor. Thus, an additional embodiment of the invention relates to the administration of sm-active compounds for the treatment or prevention of premature labor.

Sm-active compounds may also be used to relax fallopian tube smooth muscle. Fallopian tube smooth muscle plays a role in the transport of the egg to the uterus. Thus, sm-active compounds may be used to regulate ovum transport, or to facilitate laparoscopic examination of the fallopian tubes, or to facilitate fertilization procedures.

In addition to those named above, methods and compositions of the present invention may find medical utility in, for example, the treatment of cardiovascular disorders (e.g., hypertension, chronic heart failure, left ventricular failure, stroke, cerebral vasospasm after subarachnoid injury, atherosclerotic heart disease, and retinal hemorrhage), renal disorders (e.g., renal vein thrombosis, kidney infarction, renal artery embolism, renal artery stenosis, and edema, hydronephritis), proliferative diseases or disorders (e.g., vascular stenosis, myocardial hypertrophy, hypertrophy and/or hyperplasia of conduit and/or resistance vessels, myocyte hypertrophy, and fibroblast proliferative diseases), inflammatory diseases (e.g., SIRS (systemic Inflammatory Response Syndromes), sepsis, polytrauma, inflammatory bowl disease, acute and chronic pain, rheumatoid arthritis, and osteoarthritis), allergic disorders (e.g., asthma, adult respiratory distress syndrome, wound healing, and scar formation), as well as several other disorders and/or diseases (e.g., periodontal disease, dysmenorrhea, premature labor, brain edema following focal injury, diffuse axonal injury, and reperfusion injury).

In certain embodiments, the present invention provides methods of treating an individual suffering from a disease (disorder or condition) that is related to vasorexalation through administering to the individual a therapeutically effective amount of a vasoactive therapeutic agent as described above. In other embodiments, the invention provides methods of preventing or reducing the onset of a vasorelaxation-related disease in an individual through administering to the individual an effective amount of a vasoactive therapeutic agent of the invention. These methods are particularly aimed at therapeutic and prophylactic treatments of animals, and more particularly, humans.

In certain embodiments, methods and compositions of the present invention are performed on a subject who has undergone, is undergoing, or will undergo a procedure selected from the group consisting of angioplasty, vascular stent placement, endarterectomy, atherectomy, bypass surgery (such as coronary artery bypass surgery; peripheral vascular bypass surgeries), vascular grafting, organ transplant, prosthetic device implanting, microvascular reconstructions, plastic surgical flap construction, and catheter emplacement.

In a specific embodiment, methods and compositions of the present invention can be used in treating or preventing airway diseases or conditions. Agents disclosed in the application can be identified to specifically target these airway-specific 14-3-3 isoforms to lead to bronchorelaxation in the airway. Exemplary airway diseases and conditions include, but are not limited to, asthma, chronic obstructive pulmonary disease (COPD), allergic rhinitis, cystic fibrosis (CF), dispnea, emphysema, wheezing, pulmonary hypertension, pulmonary fibrosis, hyper-responsive airways, chronic bronchitis, bronchoconstriction, difficult breathing, impeded or obstructed lung airways, pulmonary vasoconstriction, impeded respiration, Acute Respiratory Distress Syndrome (ARDS), infantile Respiratory Distress Syndrome (infantile RDS), and decreased lung surfactant. While we do not wish to be bound by theory, agents disclosed herein may mediate relaxation in the airway by specifically targeting airway-specific 14-3-3 isoforms (Qi and Martinez, 2003, Radiat Res. 2003, 160(2):217-23).

Asthma is a condition that affects the airways, primarily the small tubes that carry air in and out of the lungs. Those who suffer asthma have airways that are almost always inflamed (red) and sensitive. Compounds of the invention may be useful in the treatment of both atopic and non-atopic asthma. The term “atopy” refers to a genetic predisposition toward the development of type I (immediate) hypersensitivity reactions against common environmental antigens. Accordingly, the expression “atopic asthma” as used herein is intended to be synonymous with “allergic asthma” (e.g., bronchial asthma which is an allergic manifestation in a sensitized person). The term “non-atopic asthma” as used herein is intended to refer to all other asthmas, especially essential or “true” asthma, which is provoked by a variety of factors, including vigorous exercise, irritant particles, and psychologic stresses.

COPD is characterized by inflammation of the airways, as is the case with asthma, but the inflammatory cells that have been found in the bronchioalveolar lavage fluid and sputum of patients are neutrophils rather than eosinophils, resulting in irreversible and progressive airways obstruction. COPD also presents itself clinically by with a wide range of variation from simple chronic bronchitis without disability to patients in a severely disabled state with chronic respiratory failure. Chronic bronchitis is associated with hyperplasia and hypertrophy of the mucus secreting glands of the submucosa in the large cartilaginous airways. Goblet cell hyperplasia, mucosal and submucosal inflammatory cell infiltration, edema, fibrosis, mucus plugs and increased smooth muscle are all found in the terminal and respiratory bronchioles. The small airways are known to be a major site of airway obstruction. Emphysema is characterized by destruction of the alveolar wall and loss of lung elasticity.

In certain embodiments of such methods, one or more vasoactive therapeutic agents can be administered, together (simultaneously) or at different times (sequentially). In addition, vasoactive therapeutic agents can be administered with another type of vasoactive compounds for treating a vasorelaxation-related disease (see below, “Pharmaceutical Formulations”). The two types of compounds may be administered simultaneously or sequentially.

In certain embodiments, gene therapy may be applicable with the use of nucleic acids encoding a therapeutic polypeptide (e.g., fragments of 14-3-3, HSP20 or cofilin). Alternatively, an antisense nucleic acid or an RNAi construct can be used for reducing or inhibiting expression of a target gene involved in vasorelaxation (e.g., 14-3-3, HSP20 or cofilin). Preferably, such gene therapy is specific for cardiovascular tissues.

Certain embodiments of the invention relate to local administration of the vasoactive agent of the invention to the site of injured or damaged tissue (e.g., damaged blood vessels) for the treatment of the injured or damaged tissue. Such damage may result from the use of a medical device in an invasive procedure. For example, in treating blocked vasculature by, for example, angioplasty, damage can result to the blood vessel. Such damage may be treated by use of the subject vasoactive compounds described herein. In to addition to repair of the damaged tissue, such treatment can also be used to alleviate and/or delay re-occlusions (e.g., restenosis). The subject compounds and compositions can be locally delivered using any of the methods known to one skilled in the art, including but not limited to, a drug delivery catheter, an infusion catheter, a drug delivery guidewire, an implantable medical device, and the like. In one embodiment, all or most of the damaged area is coated with the vasoactive agent, described herein per se or in a pharmaceutically acceptable carrier or excipient which serves as a coating matrix. This coating matrix can be of a liquid, gel or semisolid consistency.

In a specific embodiment of treating cardiovascular diseases and disorders, the vasoactive agent of the invention can be administered directly to the damaged vascular or non-vascular surface intravenously by using an intra-arterial or intravenous catheter, suitable for delivery of the compounds to the desired location. The location of damaged arterial surfaces can be determined by conventional diagnostic methods, such as X-ray angiography, performed using routine and well-known methods available to one skilled in the art. In addition, administration of the vasoactive therapeutic agent, using an intra-arterial or intravenous catheter is performed using routine methods well known to one skilled in the art. Typically, the compound or composition is delivered to the site of angioplasty through the same catheter used for the primary procedure, usually introduced to the carotid or coronary artery at the time of angioplasty balloon inflation.

Depending on the nature of the disease (condition) and the therapy, administration of the vasoactive agents of the invention may be continued while the other therapy is being administered and/or thereafter. Administration of the vasoactive agents may be made in a single dose, or in multiple doses. In some instances, administration of the vasoactive agent is commenced at least several days prior to the conventional therapy, while in other instances, administration is begun either immediately before or at the time of the administration of the conventional therapy.

### VI. Pharmaceutical Formulations

In certain embodiments, therapeutic agents of the present invention are formulated with a pharmaceutically acceptable carrier. Such therapeutic agents can be administered alone or as a component of a pharmaceutical formulation (composition). The compounds may be formulated for administration in any convenient way for use in human or veterinary medicine. In certain embodiments, the compound included in the pharmaceutical preparation may itself be active, or may be a prodrug.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the sm-active agents (compounds) include those suitable for oral, pulmonary (including nasal), topical, parenteral, percutaneous intrapericardial delivery, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Methods of preparing these formulations or compositions include combining a therapeutic agent of the invention and a carrier and, optionally, one or more accessory ingredients. In general, the formulations can be prepared with a liquid carrier, or a finely divided solid carrier, or both, and then, if necessary, shaping the product.

In certain aspects, the sm-active compounds disclosed herein may be administered into the respiratory system either by inhalation, respiration, nasal administration or intrapulmonary instillation (into the lungs) of a subject by any suitable means. The respiratory tract includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli which then lead to the ultimate respiratory zone, the alveoli, or deep lung. Herein, administration by inhalation may be oral and/or nasal. Examples of pharmaceutical devices for aerosol delivery include metered dose inhalers (MDIs), dry powder inhalers (DPIs), and air-jet nebulizers. Exemplary nucleic acid delivery systems by inhalation which can be readily adapted for delivery of the subject sm-active agents are described in, for example, U.S. Pat. Nos. 5,756,353; 5,858,784; and PCT applications WO98/31346; WO98/10796; WO00/27359; WO01/54664; WO02/060412. Other aerosol formulations that may be used for delivering the sm-active agents are described in U.S. Pat. Nos. 6,294,153; 6,344,194; 6,071,497, and PCT applications WO02/066078; WO02/053190; WO01/60420; WO00/66206. Further, methods for delivering sm-active agents can be adapted from those used in delivering other small molecules by inhalation, such as described in Templin et al., Antisense Nucleic Acid Drug Dev, 2000, 10:359-68; Sandrasagra et al., Expert Opin Biol Ther, 2001, 1:979-83; Sandrasagra et al., Antisense Nucleic Acid Drug Dev, 2002, 12:177-81.

Preferably, they are administered by generating an aerosol or spray comprised of powdered or liquid nasal, intrapulmonary, respirable or inhalable particles. The respirable or inhalable particles comprising the bronchoactive compound are inhaled by the subject, for example, by inhalation or by nasal administration or by instillation into the respiratory tract or the lung itself. The formulation may comprise respirable or inhalable liquid or solid particles of the bronchoactive compound that, in accordance with the present invention, include respirable or inhalable particles of a size sufficiently small to pass through the mouth and larynx upon inhalation and continue into the bronchi and alveoli of the lungs. In general, particles ranging from about 0.05, about 0.1, about 0.5, about 1 or about 2 to about 4, about 6, about 8 or about 10 microns in size. More particularly, about 0.5 to less than about 5 microns in size, are respirable or inhalable. Particles of non-respirable size which are included in an aerosol or spray tend to deposit in the throat and be swallowed. The quantity of non-respirable particles in the aerosol is, thus, preferably minimized. For nasal administration or intrapulmonary instillation, a particle size in the range of about 8, about 10, about 20 or about 25 to about 35, about 50, about 100, about 150, about 250 or about 500 μm is preferred to ensure retention in the nasal cavity or for instillation and direct deposition into the lung. Optionally, administration by nasal aerosol or inhalation can be done through the use of a nebulizer (e.g., an air-jet nebulizer), a dry powder inhaler (DPI) or a metered dose inhaler (MDI). Liquid formulations may be squirted into the respiratory tract (nose) and the lung, particularly when administered to newborns and infants.

Liquid pharmaceutical compositions of bronchoactive compound for producing an aerosol may be prepared by combining the bronchoactive compound with a stable vehicle, such as sterile pyrogen free water. Solid particulate compositions containing respirable dry particles of micronized active compound may be prepared by grinding dry active compound with a mortar and pestle, and then passing the micronized composition through a 400 mesh screen to break up or separate out large agglomerates. A solid particulate composition comprised of the vasoactive compound may optionally contain a dispersant that serves to facilitate the formation of an aerosol. A suitable dispersant is lactose, which may be blended with the active compound in any suitable ratio, e.g., a 1 to 1 ratio by weight. Aerosols of liquid particles comprising the bronchoactive compound may be produced by any suitable means, such as with a nebulizer (see, e.g. U.S. Pat. No. 4,501,729). Nebulizers are commercially available devices which transform solutions or suspensions of the active ingredient into a therapeutic aerosol mist either by means of acceleration of a compressed gas, typically air or oxygen, through a narrow venturi orifice or by means of ultrasonic agitation. Suitable compositions for use in nebulizer consist of the active ingredient in liquid carrier, the active ingredient comprising up to 40% w/w composition, but preferably less than 20% w/w carrier being typically water or a dilute aqueous alcoholic solution, preferably made isotonic with body fluids by the addition of, for example sodium chloride. Optional additives include preservatives if the composition is not prepared sterile, for example, methyl hydroxybenzoate, anti-oxidants, flavoring agents, volatile oils, buffering agents and surfactants. Aerosols of solid particles comprising the bronchoactive compound may likewise be produced with any sold particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. Examples of such aerosol generators include metered dose inhalers and insufflators.

In certain embodiments, systemic administration can also be accomplished by inhalation or insufflation of a powder, i.e., particulate composition containing the active ingredient. For example, the active ingredient in powder form may be inhaled into the lungs using conventional devices for aerosolizing particulate formulations. The active ingredient as a particulate formulation may also be administered by insufflation, i.e., blown or otherwise dispersed into suitable body tissues or cavities by simple dusting or using conventional devices for aerosolizing particulate formulations. These particulate compositions may also be formulated to provide delayed-, sustained-, and/or controlled-release of the active ingredient in accordance with well understood principles and known materials. The human lungs can remove or rapidly degrade hydrolytically cleavable deposited aerosols over periods ranging from minutes to hours. In the upper airways, ciliated epithelia contribute to the “mucociliary excalator” by which particles are swept from the airways toward the mouth. Pavia, D., “Lung Mucociliary Clearance,” in Aerosols and the Lung: Clinical and Experimental Aspects, Clarke, S. W. and Pavia, D., Eds., Butterworths, London, 1984. In the deep lungs, alveolar macrophages are capable of phagocytosing particles soon after their deposition. The deep lung, or alveoli, are the primary target of inhaled therapeutic aerosols for systemic delivery. In situations where systemic delivery is desired, a subject sm-active compound is optionally formulated as microparticles.

In certain preferred embodiments, the aerosoled sm-active agents are formulated as microparticles. Microparticles having a diameter of between 0.5 and ten microns can penetrate the lungs, passing through most of the natural barriers. A diameter of less than ten microns is typically required to bypass the throat; a diameter of 0.5 microns or greater is typically required to avoid being exhaled. Thus, in one embodiment, microparticles of the invention have an average diameter of less than 20 microns.

In certain preferred embodiments, the subject sm-active agents are formulated in a supramolecular complex, e.g., having a diameter of between 0.5 and ten microns, which can be aggregated into particles, e.g., having a diameter of between 0.5 and ten microns.

In other embodiments, the subject sm-active agents are provided in liposomes or supramolecular complexes appropriately formulated for pulmonary delivery.

(i). Supramolecular Complexes

In certain embodiments, the subject sm-active agents are formulated as part of a “supramolecular complex.” To further illustrate, the sm-active agents can be contacted with at least one polymer to form a composite and then the polymer of the composite treated under conditions sufficient to form a supramolecular complex containing the sm-active agents and a multi-dimensional polymer network. The polymer molecule may be linear or branched. Accordingly, a group of two or more polymer molecules may be linear, branched, or a mixture of linear and branched polymers. The composite may be prepared by any suitable means known in the art. For example, the composite may be formed by simply contacting, mixing or dispersing the sm-active agents with a polymer (e.g., a cyclodextrin-modified polymer). A composite may also be prepared by polymerizing monomers, which may be the same or different, capable of forming a linear or branched polymer in the presence of the sm-active agents. The composite may be further modified with at least one ligand, e.g., to direct cellular uptake of the sm-active agents or otherwise effect tissue or cellular distribution in vivo of the sm-active agents. The composite may take any suitable form and, preferably, is in the form of particles.

In certain preferred embodiments, the subject sm-active agents are formulated with β-cyclodextrin containing polymers (βCD-polymers). βCD-polymers are capable of forming polyplexes with certain small organic agents. The βCD-polymers can be synthesized, for instance, by the condensation of a diamino-cyclodextrin monomer A with a diimidate comonomer B. Cyclodextrins are cyclic polysaccharides containing naturally occurring D(+)-glucopyranose units in an α-(1,4) linkage. The most common cyclodextrins are β-cyclodextrins, 1-cyclodextrins and γ-cyclodextrins which contain, respectively, six, seven or eight glucopyranose units. Exemplary cyclodextrin delivery systems which can be readily adapted for delivery of the subject sm-active agents are described in, for example, the Gonzalez et al PCT application WO00/01734 and Davis PCT application WO00/33885.

In certain embodiments, the supramolecular complexes are aggregated into particles, for example, formulations of particles having an average diameter of between 20 and 500 nanometer (nm), and even more preferably, between 20 and 200 nm.

(ii). Polymers for Forming Microparticles

In addition to the supramolecular complexes described above, a number of other polymers can be used to form the microparticles. As used herein, the term “microparticles” includes microspheres (uniform spheres), microcapsules (having a core and an outer layer of polymer), and particles of irregular shape.

Polymers are preferably biodegradable within the time period over which release of the sm-active agents is desired or relatively soon thereafter, generally in the range of one year, more typically a few months, even more typically a few days to a few weeks. Biodegradation can refer to either a breakup of the microparticle, that is, dissociation of the polymers forming the microparticles and/or of the polymers themselves. This can occur as a result of change in pH from the carrier in which the particles are administered to the pH at the site of release, as in the case of the diketopiperazines, hydrolysis, as in the case of poly(hydroxy acids), by diffusion of an ion such as calcium out of the microparticle, as in the case of microparticles formed by ionic bonding of a polymer such as alginate, and by enzymatic action, as in the case of many of the polysaccharides and proteins. In some cases linear release may be most useful, although in others a pulse release or “bulk release” may provided more effective results.

Representative synthetic materials are: diketopiperazines, poly(hydroxy acids) such as poly(lactic acid), poly(glycolic acid) and copolymers thereof, polyanhydrides, polyesters such as polyorthoesters, polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, polyvinyl esters, polyvinyl halides, polyvinylpyrrolidone, polyvinylacetate, and poly vinyl chloride, polystyrene, polysiloxanes, polymers of acrylic and methacrylic acids including poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyurethanes and co-polymers thereof, celluloses including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt, poly(butic acid), poly(valeric acid), and poly(lactide-co-caprolactone).

Natural polymers include alginate and other polysaccharides including dextran and cellulose, collagen, albumin and other hydrophilic proteins, zein and other prolamines and hydrophobic proteins, copolymers and mixtures thereof. As used herein, chemical derivatives thereof refer to substitutions, additions of chemical groups, for example, alkyl, alkylene, hydroxylations, oxidations, and other modifications routinely made by those skilled in the art.

Bioadhesive polymers include bioerodible hydrogels described by H. S. Sawhney, C. P. Pathak and J. A. Hubell in Macromolecules, 1993, 26, 581-587, polyhyaluronic acids, casein, gelatin, glutin, polyanhydrides, polyacrylic acid, alginate, chitosan, and polyacrylates.

To further illustrate, the matrices can be formed of the polymers by solvent evaporation, spray drying, solvent extraction and other methods known to those skilled in the art. Methods developed for making microspheres for drug delivery are described in the literature, for example, as described by Mathiowitz and Langer, J. Controlled Release 5, 13-22 (1987); Mathiowitz, et al., Reactive Polymers 6, 275-283 (1987); and Mathiowitz, et al., J. Appl. Polymer Sci. 35, 755-774 (1988). The selection of the method depends on the polymer selection, the size, external morphology, and crystallinity that is desired, as described, for example, by Mathiowitz, et al., Scanning Microscopy 4, 329-340 (1990); Mathiowitz, et al., J. Appl. Polymer Sci. 45, 125-134 (1992); and Benita, et al., J. Pharm. Sci. 73, 1721-1724 (1984).

In solvent evaporation, described for example, in Mathiowitz, et al., (1990), Benita, and U.S. Pat. No. 4,272,398 to Jaffe, the polymer is dissolved in a volatile organic solvent. The sm-active agent, either in soluble form or dispersed as fine particles, is added to the polymer solution, and the mixture is suspended in an aqueous phase that contains a surface active agent such as poly(vinyl alcohol). The resulting emulsion is stirred until most of the organic solvent evaporates, leaving solid microspheres.

In general, the polymer can be dissolved in methylene chloride. Several different polymer concentrations can be used, for example, between 0.05 and 0.20 g/ml. After loading the solution with drug, the solution is suspended in 200 ml of vigorously stirring distilled water containing 1% (w/v) poly(vinyl alcohol) (Sigma Chemical Co., St. Louis, Mo.). After four hours of stirring, the organic solvent will have evaporated from the polymer, and the resulting microspheres will be washed with water and dried overnight in a lyophilizer or simply dried.

Microspheres with different sizes (1-1000 microns, though less than 10 microns for aerosol applications) and morphologies can be obtained by this method which is useful for relatively stable polymers such as polyesters and polystyrene. However, labile polymers such as polyanhydrides may degrade due to exposure to water. For these polymers, hot melt encapsulation and solvent removal may be preferred.

In hot melt encapsulation, the polymer is first melted and then mixed with the solid particles of sm-active agent, preferably sieved to appropriate size. The mixture is suspended in a non-miscible solvent such as silicon oil and, with continuous stirring, heated to 5° C. above the melting point of the polymer. Once the emulsion is stabilized, it is cooled until the polymer particles solidify. The resulting microspheres are washed by decantation with petroleum ether to give a free-flowing powder. Microspheres with diameters between one and 1000 microns can be obtained with this method. The external surface of spheres prepared by this technique are usually smooth and dense. This procedure is useful with water labile polymers, but is limited to use with polymers with molecular weights between 1000 and 50000.

In spray drying, the polymer is dissolved in an organic solvent such as methylene chloride (0.04 g/ml). A known amount of sm-active agent is suspended (if insoluble) or co-dissolved (if soluble) in the polymer solution. The solution or the dispersion is then spray-dried. Microspheres ranging in diameter between one and ten microns can be obtained with a morphology which depends on the selection of polymer.

Hydrogel microspheres made of gel-type polymers such as alginate or polyphosphazines or other dicarboxylic polymers can be prepared by dissolving the polymer in an aqueous solution, suspending the material to be incorporated into the mixture, and extruding the polymer mixture through a microdroplet forming device, equipped with a nitrogen gas jet. The resulting microspheres fall into a slowly stirring, ionic hardening bath, as described, for example, by Salib, et al., Pharmazeutische Industrie 40-111A, 1230 (1978). The advantage of this system is the ability to further modify the surface of the microspheres by coating them with polycationic polymers such as polylysine, after fabrication, for example, as described by Lim, et al., J. Pharm. Sci. 70, 351-354 (1981). For example, in the case of alginate, a hydrogel can be formed by ionically crosslinking the alginate with calcium ions, then crosslinking the outer surface of the microparticle with a polycation such as polylysine, after fabrication. The microsphere particle size will be controlled using various size extruders, polymer flow rates and gas flow rates.

Chitosan microspheres can be prepared by dissolving the polymer in acidic solution and crosslinking with tripolyphosphate. For example, carboxymethylcellulose (CMC) microsphere are prepared by dissolving the polymer in an acid solution and precipitating the microspheres with lead ions. Alginate/polyethyleneimine (PEI) can be prepared to reduce the amount of carboxyl groups on the alginate microcapsules. Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic agents of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In particular, compositions of the invention can be administered topically, either to skin or to mucosal membranes. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required. The ointments, pastes, creams and gels may contain, in addition to a vasoactive agent, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a therapeutic agent, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more therapeutic agents in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable forms are made by forming microencapsule matrices of one or more therapeutic agents in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In certain embodiments, the subject methods of the invention can be used alone. Alternatively, the subject methods may be used in combination with other conventional therapeutic approaches directed to modulate smooth muscle tone and treat vasorelaxation-related diseases such as restenosis and atherosclerosis and bronochorelaxation-related diseases such as asthma. For example, such methods can be used in combination with other conventional sm-active compounds. The present invention recognizes that the effectiveness of conventional sm-active compounds can be enhanced through the use of a sm-active therapeutic agent of the invention (as described above).

A wide array of conventional compounds has been shown to have sm-active (e.g., vasoactive or bronchoactive) activities. These compounds have been used as pharmaceutical agents to modulate smooth muscle tone (e.g., relax or constrict vessels). It has been shown that when two or more different treatments are combined, the treatments may work synergistically and allow reduction of dosage of each of the treatments, thereby reducing the possible detrimental side effects exerted by each compound at higher dosages. When a therapeutic agent of the present invention is administered in combination with another conventional sm-active compound, either concomitantly or sequentially, such therapeutic agent is shown to enhance the therapeutic effect of the subject agent or overcome cellular resistance to such agent. This allows decrease of dosage of a sm-active agent, thereby reducing the undesirable side effects, or restores the effectiveness of a sm-active agent in resistant cells.

Suitable conventional pharmaceutical compounds that may be used for such conjoint therapy include, but are not limited to, potassium channel activators, calcium channel blockers, beta-blockers, long and short acting alpha-adrenergic receptor antagonists, prostaglandins, phosphodiesterase inhibitors, adenosine, ergot alkaloids, vasoactive intestinal peptides, dopamine agonists, opioid antagonists, endothelin antagonists, thromboxane inhibitors and the like.

For example, conventional pharmaceutical compounds include, but are not limited to, nitric oxide donors; antithrombogenic agents (for example, heparin, covalent heparin, hirudin, hirulog, coumadin, protamine, argatroban, D-phenylalanyl-L-poly-L-arginyl chloromethyl ketone, and the like); thrombolytic agents (for example, urokinase, streptokinase, tissue plasminogen activators, and the like); fibrinolytic agents; vasospasm inhibitors; potassium channel activators (for example, nicorandil, pinacidil, cromakalim, minoxidil, aprilkalim, loprazolam and the like); calcium channel blockers (for example, nifedipine, verapamil, diltiazem, gallopamil, niludipine, nimodipins, nicardipine, and the like); antihypertensive agents (for example, HYTRIN®, and the like); antimicrobial agents or antibiotics (for example, adriamycin, and the like); antiplatelet agents (for example, aspirin, ticlopidine, a glycoprotein IIb/IIIa inhibitor, surface glycoprotein receptors and the like); antimitotic, antiproliferative agents or microtubule inhibitors (for example, taxanes, colchicine, methotrexate, azathioprine, vincristine, vinblastine, cytochalasin, fluorouracil, adriamycin, mutamycin, tubercidin, epothilone A or B, discodermolide, and the like); antisecretory agents (such as, for example, retinoid, and the like); remodelling inhibitors; antisense nucleotides (for example, deoxyribonucleic acid, and the like); anti-cancer agents (for example, tamoxifen citrate, acivicin, bizelesin, daunorubicin, epirubicin, mitoxantrone, and the like); steroids (for example, dexamethasone, dexamethasone sodium phosphate, dexamethasone acetate, β-estradiol, and the like); non-steroidal anti-inflammatory agents (NSAID); COX-2 inhibitors; 5-lipoxygenase (5-LO) inhibitors; leukotriene B4 (LTB4) receptor antagonists; leukotriene A4 (LTA4) hydrolase inhibitors; 5-HT agonists; HMG-CoA inhibitors; H2 receptor antagonists; antineoplastic agents, thromboxane inhibitors; decongestants; diuretics; sedating or non-sedating anti-histamines; inducible nitric oxide synthase inhibitors; opioids, analgesics; proton pump inhibitors; isoprostane inhibitors; vasoactive agents; B-agonists; anticholinergic; mast cell stabilizer; immunosuppressive agents (for example cyclosporin, rapamycin, everolimus, actinomycin D and the like); growth factor antagonists or antibodies (for example, trapidal (a PDGF antagonist), angiopeptin (a growth hormone antagonist), angiogenin, and the like); dopamine agonists (for example, apomorphine, bromocriptine, testosterone, cocaine, strychnine, and the like); biologic agents (for example, peptides, proteins, enzymes, extracellular matrix components, cellular components, and the like); angiotensin converting enzyme (ACE) inhibitors; angiotensin II receptor antagonists; renin inhibitors; free radical scavengers, iron chelators or antioxidants (for example, ascorbic acid, alpha tocopherol, superoxide dismutase, deferoxamine, 21-aminosteroid, and the like); sex hormones (for example, estrogen, and the like); antipolymerases (for example, AZT, and the like); antiviral agents (for example, acyclovir, famciclovir, rimantadine hydrochloride, ganciclovir sodium, Norvir®, Crixivan®, and the like); photodynamic therapy agents (for example, 5-aminolevulinic acid, meta-tetrahydroxyphenylchlorin, hexadecafluoro zinc phthalocyanine, tetramethyl hematoporphyrin, rhodamine 123, and the like); antibody targeted therapy agents; and gene therapy agent.

As another example, conventional pharmaceutical compounds include other bioactive agents such as the currently prescribed drugs for asthma, COPD, and allergic rhinitis. These include β-2 adrenergic agonists such as ephedrine, isoproterenol, isoetharine, epinephrine, metaproterenol, terbutaline, fenoterol, procaterol, albuterol, salbutamol, pirbuterol, formoterol, biloterol, bambuterol, salmeterol and seretide, among others; other anti-cholinergic agents; anti-histaminic agents; adenosine A1, A2b and A3 receptor antagonists such as anti-sense oligos, among others; adenosine A2a agonists; and glucocorticosteroids.

In a further embodiment, compositions of the present invention further include one or more agents selected from immune response modifiers, anti-proliferatives, corticosteroids, angiostatic steroids, anti parasitic drugs, anti glaucoma drugs, antibiotics, antisense compounds, differentiation modulators, antiviral drugs, anticancer drugs, and non-steroidal anti-inflammatory drugs.

### VII. Medical Device Coatings

Another aspect of the invention relates to coated medical devices. For instance, in certain embodiments, the subject invention provides a medical device having a coating adhered to at least one surface, wherein the coating includes the subject polymer matrix and a sm-active agent of the present invention. Such coatings can be applied to surgical implements such as screws, plates, washers, sutures, prosthesis anchors, tacks, staples, electrical leads, valves, membranes. The devices can be catheters, implantable vascular access ports, blood storage bags, blood tubing, central venous catheters, arterial catheters, vascular grafts, intraaortic balloon pumps, heart valves, cardiovascular sutures, artificial hearts, a pacemaker, ventricular assist pumps, extracorporeal devices, blood filters, hemodialysis units, hemoperfusion units, plasmapheresis units, and filters adapted for deployment in a blood vessel.

In some embodiments according to the present invention, monomers for forming a polymer are combined with a sm-active agent and are mixed to make a homogeneous dispersion of the sm-active agent in the monomer solution. The dispersion is then applied to a stent or other device according to a conventional coating process, after which the crosslinking process is initiated by a conventional initiator, such as UV light. In other embodiments according to the present invention, a polymer composition is combined with a sm-active agent to form a dispersion. The dispersion is then applied to a surface of a medical device and the polymer is cross-linked to form a solid coating. In other embodiments according to the present invention, a polymer and a sm-active agent are combined with a suitable solvent to form a dispersion, which is then applied to a stent in a conventional fashion. The solvent is then removed by a conventional process, such as heat evaporation, with the result that the polymer and sm-active agent (together forming a sustained-release drug delivery system) remain on the stent as a coating. An analogous process may be used where the sm-active agent is dissolved in the polymer composition.

In some embodiments according to the invention, the system comprises a polymer that is relatively rigid. In other embodiments, the system comprises a polymer that is soft and malleable. In still other embodiments, the system includes a polymer that has an adhesive character. Hardness, elasticity, adhesive, and other characteristics of the polymer are widely variable, depending upon the particular final physical form of the system, as discussed in more detail below.

Embodiments of the system according to the present invention take many different forms. In some embodiments, the system consists of the sm-active agent suspended or dispersed in the polymer. In certain other embodiments, the system consists of a sm-active agent and a semi solid or gel polymer, which is adapted to be injected via a syringe into a body. In other embodiments according to the present invention, the system consists of a sm-active agent and a soft flexible polymer, which is adapted to be inserted or implanted into a body by a suitable surgical method. In still further embodiments according to the present invention, the system consists of a hard, solid polymer, which is adapted to be inserted or implanted into a body by a suitable surgical method. In further embodiments, the system comprises a polymer having the sm-active agent suspended or dispersed therein, wherein the sm-active agent and polymer mixture forms a coating on a surgical implement, such as a screw, stent, pacemaker, etc. In particular embodiments according to the present invention, the device consists of a hard, solid polymer, which is shaped in the form of a surgical implement such as a surgical screw, plate, stent, etc., or some part thereof. In other embodiments according to the present invention, the system includes a polymer that is in the form of a suture having the sm-active agent dispersed or suspended therein.

In some embodiments according to the present invention, provided is a medical device comprising a substrate having a surface, such as an exterior surface that is contact with or proximal to vascular tissue, and a coating on the exterior surface. The coating comprises a polymer and a sm-active agent dispersed in the polymer, wherein the polymer is permeable to the sm-active agent or biodegrades to release the sm-active agent. In certain embodiments according to the present invention, the device comprises a sm-active agent suspended or dispersed in a suitable polymer, wherein the sm-active agent and polymer are coated onto an entire substrate, e.g., a surgical implement. Such coating may be accomplished by spray coating or dip coating.

In other embodiments according to the present invention, the device comprises a sm-active agent and polymer suspension or dispersion, wherein the polymer is rigid, and forms a constituent part of a device to be inserted or implanted into a body, e.g., where that part of the device is in contact with or proximal to vascular tissue. For instance, in particular embodiments according to the present invention, the device is a surgical screw, stent, pacemaker, etc. coated with the sm-active agent suspended or dispersed in the polymer. In other particular embodiments according to the present invention, the polymer in which the sm-active agent is suspended forms a tip or a head, or part thereof. In other embodiments according to the present invention, the polymer in which sm-active agent is suspended or dispersed, is coated onto a surgical implement such as surgical tubing (such as colostomy, peritoneal lavage, catheter, and intravenous tubing). In still further embodiments according to the present invention, the device is an intravenous needle having the polymer and sm-active agent coated thereon.

As discussed above, the coating according to the present invention comprises a polymer that is bioerodible or non-bioerodible. The choice of bioerodible versus non-bioerodible polymer is made based upon the intended end use of the system or device. In some embodiments according to the present invention, the polymer is advantageously bioerodible. For instance, where the system is a coating on a surgically implantable device, such as a screw, stent, pacemaker, etc., the polymer is advantageously bioerodible. Other embodiments according to the present invention in which the polymer is advantageously bioerodible include devices that are implantable, inhalable, or injectable suspensions or dispersions of a sm-active agent in a polymer, wherein the further elements (such as screws or anchors) are not utilized.

In some embodiments according to the present invention wherein the polymer is poorly permeable and bioerodible, the rate of bioerosion of the polymer is advantageously sufficiently slower than the rate of sm-active agent release so that the polymer remains in place for a substantial period of time after the sm-active agent has been released, but is eventually bioeroded and resorbed into the surrounding tissue. For example, where the device is a bioerodible suture comprising the sm-active agent suspended or dispersed in a bioerodible polymer, the rate of bioerosion of the polymer is advantageously slow enough that the sm-active agent is released in a linear manner over a period of about three to about 14 days, but the sutures persist for a period of about three weeks to about six months. Similar devices according to the present invention include surgical staples comprising a sm-active agent suspended or dispersed in a bioerodible polymer.

In other embodiments according to the present invention, the rate of bioerosion of the polymer is advantageously on the same order as the rate of sm-active agent release. For instance, where the system comprises a vasoactive agent suspended or dispersed in a polymer that is coated onto a surgical implement, such as an orthopedic screw, a stent, a pacemaker, or a non-bioerodible suture, the polymer advantageously bioerodes at such a rate that the surface area of the vasoactive agent that is directly exposed to the surrounding body tissue remains substantially constant over time.

In other embodiments according to the present invention, the polymer vehicle is permeable to water in the surrounding tissue, e.g., in blood plasma. In such cases, water solution may permeate the polymer, thereby contacting the sm-active agent. The rate of dissolution may be governed by a complex set of variables, such as the polymer's permeability, the solubility of the sm-active agent, the pH, ionic strength, and protein composition, etc. of the physiologic fluid. In some embodiments according to the present invention, the polymer is non-bioerodible. Non-bioerodible polymers are especially useful where the system includes a polymer intended to be coated onto, or form a constituent part, of a surgical implement that is adapted to be permanently, or semi-permanently, inserted or implanted into a body. Exemplary devices in which the polymer advantageously forms a permanent coating on a surgical implement include an orthopedic screw, a stent, a prosthetic joint, an artificial valve, a permanent suture, a pacemaker, etc.

There is a multiplicity of different stents that may be utilized following percutaneous transluminal coronary angioplasty. Although any number of stents may be utilized in accordance with the present invention, for simplicity, a limited number of stents will be described in exemplary embodiments of the present invention. The skilled artisan will recognize that any number of stents may be utilized in connection with the present invention. In addition, as stated above, other medical devices may be utilized.

A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

The stents of the present invention may be fabricated utilizing any number of methods. For example, the stent may be fabricated from a hollow or formed stainless steel tube that may be machined using lasers, electric discharge milling, chemical etching or other means. The stent is inserted into the body and placed at the desired site in an unexpanded form. In one exemplary embodiment, expansion may be effected in a blood vessel by a balloon catheter, where the final diameter of the stent is a function of the diameter of the balloon catheter used.

It should be appreciated that a stent in accordance with the present invention may be embodied in a shape-memory material, including, for example, an appropriate alloy of nickel and titanium or stainless steel.

Structures formed from stainless steel may be made self-expanding by configuring the stainless steel in a predetermined manner, for example, by twisting it into a braided configuration. In this embodiment after the stent has been formed it may be compressed so as to occupy a space sufficiently small as to permit its insertion in a blood vessel or other tissue by insertion means, wherein the insertion means include a suitable catheter, or flexible rod.

On emerging from the catheter, the stent may be configured to expand into the desired configuration where the expansion is automatic or triggered by a change in pressure, temperature or electrical stimulation.

Regardless of the design of the stent, it is preferable to have the sm-active agent applied with enough specificity and a sufficient concentration to provide an effective dosage in the lesion area. In this regard, the “reservoir size” in the coating is preferably sized to adequately apply the sm-active agent at the desired location and in the desired amount.

In an alternate exemplary embodiment, the entire inner and outer surface of the stent may be coated with the sm-active agent in therapeutic dosage amounts. It is, however, important to note that the coating techniques may vary depending on the sm-active agent. Also, the coating techniques may vary depending on the material comprising the stent or other intraluminal medical device.

The intraluminal medical device comprises the sustained release drug delivery coating. The sm-active agent coating may be applied to the stent via a conventional coating process, such as impregnating coating, spray coating and dip coating.

In one embodiment, an intraluminal medical device comprises an elongated radially expandable tubular stent having an interior luminal surface and an opposite exterior surface extending along a longitudinal stent axis. The stent may include a permanent implantable stent, an implantable grafted stent, or a temporary stent, wherein the temporary stent is defined as a stent that is expandable inside a vessel and is thereafter retractable from the vessel. The stent configuration may comprise a coil stent, a memory coil stent, a Nitinol stent, a mesh stent, a scaffold stent, a sleeve stent, a permeable stent, a stent having a temperature sensor, a porous stent, and the like. The stent may be deployed according to conventional methodology, such as by an inflatable balloon catheter, by a self-deployment mechanism (after release from a catheter), or by other appropriate means. The elongate radially expandable tubular stent may be a grafted stent, wherein the grafted stent is a composite device having a stent inside or outside of a graft. The graft may be a vascular graft, such as an ePTFE graft, a biological graft, or a woven graft.

The sm-active agent may be incorporated onto or affixed to the stent in a number of ways. In the exemplary embodiment, the sm-active agent is directly incorporated into a polymeric matrix and sprayed onto the outer surface of the stent. The sm-active agent elutes from the polymeric matrix over time and enters the surrounding tissue. The sm-active agent preferably remains on the stent for at least three days up to approximately six months, and more preferably between seven and thirty days.

In certain embodiments, the polymer according to the present invention comprises any biologically tolerated polymer that is permeable to the sm-active agent and while having a permeability such that it is not the principal rate determining factor in the rate of release of the sm-active agent from the polymer.

In some embodiments according to the present invention, the polymer is non-bioerodible. Examples of non-bioerodible polymers useful in the present invention include poly(ethylene-co-vinyl acetate) (EVA), polyvinylalcohol and polyurethanes, such as polycarbonate-based polyurethanes. In other embodiments of the present invention, the polymer is bioerodible. Examples of bioerodible polymers useful in the present invention include polyanhydride, polylactic acid, polyglycolic acid, polyorthoester, polyalkylcyanoacrylate or derivatives and copolymers thereof. The skilled artisan will recognize that the choice of bioerodibility or non-bioerodibility of the polymer depends upon the final physical form of the system, as described in greater detail below. Other exemplary polymers include polysilicone and polymers derived from hyaluronic acid. The skilled artisan will understand that the polymer according to the present invention is prepared under conditions suitable to impart permeability such that it is not the principal rate determining factor in the release of the sm-active agent from the polymer.

Moreover, suitable polymers include naturally occurring (collagen, hyaluronic acid, etc.) or synthetic materials that are biologically compatible with bodily fluids and mammalian tissues, and essentially insoluble in bodily fluids with which the polymer will come in contact. In addition, the suitable polymers essentially prevent interaction between the sm-active agent dispersed/suspended in the polymer and proteinaceous components in the bodily fluid. The use of rapidly dissolving polymers or polymers highly soluble in bodily fluid or which permit interaction between the sm-active agent and proteinaceous components are to be avoided in certain instances since dissolution of the polymer or interaction with proteinaceous components would affect the constancy of drug release.

Other suitable polymers include polypropylene, polyester, polyethylene vinyl acetate (PVA or EVA), polyethylene oxide (PEO), polypropylene oxide, polycarboxylic acids, polyalkylacrylates, cellulose ethers, silicone, poly(dl-lactide-co glycolide), various Eudragrits (for example, NE30D, RS PO and RL PO), polyalkyl-alkylacrylate copolymers, polyester-polyurethane block copolymers, polyether-polyurethane block copolymers, polydioxanone, poly-(-hydroxybutyrate), polylactic acid (PLA), polycaprolactone, polyglycolic acid, and PEO-PLA copolymers.

The coating of the present invention may be formed by mixing one or more suitable monomers and a suitable sm-active agent, then polymerizing the monomer to form the polymer system. In this way, the sm-active agent is dissolved or dispersed in the polymer. In other embodiments, the sm-active agent is mixed into a liquid polymer or polymer dispersion and then the polymer is further processed to form the inventive coating. Suitable further processing may include crosslinking with suitable crosslinking sm-active agents, further polymerization of the liquid polymer or polymer dispersion, copolymerization with a suitable monomer, block copolymerization with suitable polymer blocks, etc. The further processing traps the sm-active agent in the polymer so that the sm-active agent is suspended or dispersed in the polymer vehicle.

Any number of non-erodible polymers may be utilized in conjunction with the sm-active agent. Film-forming polymers that can be used for coatings in this application can be absorbable or non-absorbable and must be biocompatible to minimize irritation to the vessel wall. The polymer may be either biostable or bioabsorbable depending on the desired rate of release or the desired degree of polymer stability, but a bioabsorbable polymer may be preferred since, unlike biostable polymer, it will not be present long after implantation to cause any adverse, chronic local response. Furthermore, bioabsorbable polymers do not present the risk that over extended periods of time there could be an adhesion loss between the stent and coating caused by the stresses of the biological environment that could dislodge the coating and introduce further problems even after the stent is encapsulated in tissue.

Suitable film-forming bioabsorbable polymers that could be used include polymers selected from the group consisting of aliphatic polyesters, poly(amino acids), copoly(ether-esters), polyalkylene oxalates, polyamides, poly(iminocarbonates), polyorthoesters, polyoxaesters, polyamidoesters, polyoxaesters containing amido groups, poly(anhydrides), polyphosphazenes, biomolecules and blends thereof. For the purpose of this invention aliphatic polyesters include homopolymers and copolymers of lactide (which includes lactic acid d-,l- and meso lactide), caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, 22:993-1009, 1988 by Cohn and Younes and Cohn, Polymer Preprints (ACS Division of Polymer Chemistry) 30(1):498, 1989 (e.g., PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein). Polyphosphazenes, co-, ter- and higher order mixed monomer based polymers made from L-lactide, D,L-lactide, lactic acid, glycolide, glycolic acid, para-dioxanone, trimethylene carbonate and caprolactone are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein). Polyanhydrides from diacids of the form HOOC—C6H4-O—(CH2)m-O—C6H4—COOH where m is an integer in the range of from 2 to 8 and copolymers thereof with aliphatic alpha-omega diacids of up to 12 carbons. Polyoxaesters, polyoxaamides and polyoxaesters containing amines and/or amido groups are described in one or more of U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference). Film-forming polymeric biomolecules for the purpose of this invention include naturally occurring materials that may be enzymatically degraded in the human body or are hydrolytically unstable in the human body such as fibrin, fibrinogen, collagen, elastin, and absorbable biocompatable polysaccharides such as chitosan, starch, fatty acids (and esters thereof), glucoso-glycans and hyaluronic acid.

Suitable film-forming biostable polymers with relatively low chronic tissue response, such as polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters could also be used. Other polymers could also be used if they can be dissolved, cured or polymerized on the stent. These include polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile, polyvinyl ketones; polyvinyl aromatics such as polystyrene; polyvinyl esters such as polyvinyl acetate; copolymers of vinyl monomers with each other and olefins, such as etheylene-methyl methacrylate copolymers, acrylonitrile-styrene copolymers, ABS resins and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e., carboxymethyl cellulose and hydroxyalkyl celluloses); and combinations thereof. Polyamides for the purpose of this application would also include polyamides of the form —NH—(CH2)ₙ-CO— and NH—(CH2)ₓ—NH—CO—(CH2)_{y}-CO, wherein n is preferably an integer from 6 to 13; x is an integer in the range from 6 to 12; and y is an integer in the range from 4 to 16. The list provided above is illustrative but not limiting.

The polymers used for coatings can be film-forming polymers that have molecular weight high enough as to not be waxy or tacky. The polymers also should adhere to the stent and should not be so readily deformable after deposition on the stent as to be able to be displaced by hemodynamic stresses. The polymers molecular weight should be high enough to provide sufficient toughness so that the polymers will not to be rubbed off during handling or deployment of the stent and must not crack during expansion of the stent. In certain embodiments, the polymer has a melting temperature above 40° C., preferably above about 45° C., more preferably above 50° C. and most preferably above 55° C. A coating may be formulated by mixing one or more of the sm-active agents with the coating polymers in a coating mixture. The sm-active agent may be present as a liquid, a finely divided solid, or any other appropriate physical form. Optionally, the mixture may include one or more additives, e.g., nontoxic auxiliary substances such as diluents, carriers, excipients, stabilizers or the like. Other suitable additives may be formulated with the polymer and sm-active agent. For example, hydrophilic polymers selected from the previously described lists of biocompatible film forming polymers may be added to a biocompatible hydrophobic coating to modify the release profile (or a hydrophobic polymer may be added to a hydrophilic coating to modify the release profile). One example would be adding a hydrophilic polymer selected from the group consisting of polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the therapeutic sm-active agents.

The thickness of the coating can determine the rate at which the sm-active agent elutes from the matrix. Essentially, the sm-active agent elutes from the matrix by diffusion through the polymer matrix. Polymers are permeable, thereby allowing solids, liquids and gases to escape therefrom. The total thickness of the polymeric matrix is in the range from about one micron to about twenty microns or greater. It is important to note that primer layers and metal surface treatments may be utilized before the polymeric matrix is affixed to the medical device. For example, acid cleaning, alkaline (base) cleaning, salinization and parylene deposition may be used as part of the overall process described.

To further illustrate, a poly(ethylene-co-vinylacetate), polybutylmethacrylate and sm-active agent solution may be incorporated into or onto the stent in a number of ways. For example, the solution may be sprayed onto the stent or the stent may be dipped into the solution. Other methods include spin coating and RF plasma polymerization. In one exemplary embodiment, the solution is sprayed onto the stent and then allowed to dry. In another exemplary embodiment, the solution may be electrically charged to one polarity and the stent electrically changed to the opposite polarity. In this manner, the solution and stent will be attracted to one another. In using this type of spraying process, waste may be reduced and more precise control over the thickness of the coat may be achieved.

In another exemplary embodiment, the sm-active agent may be incorporated into a film-forming polyfluoro copolymer comprising an amount of a first moiety selected from the group consisting of polymerized vinylidenefluoride and polymerized tetrafluoroethylene, and an amount of a second moiety other than the first moiety and which is copolymerized with the first moiety, thereby producing the polyfluoro copolymer, the second moiety being capable of providing toughness or elastomeric properties to the polyfluoro copolymer, wherein the relative amounts of the first moiety and the second moiety are effective to provide the coating and film produced therefrom with properties effective for use in treating implantable medical devices.

In one embodiment according to the present invention, the exterior surface of the expandable tubular stent of the intraluminal medical device of the present invention comprises a coating according to the present invention. The exterior surface of a stent having a coating is the tissue-contacting surface and is biocompatible. The “sustained release sm-active agent delivery system coated surface” is synonymous with “coated surface,” which surface is coated, covered or impregnated with a sustained release sm-active agent delivery system according to the present invention.

In an alternate embodiment, the interior luminal surface or entire surface (i.e., both interior and exterior surfaces) of the elongate radially expandable tubular stent of the intraluminal medical device of the present invention has the coated surface. The interior luminal surface having the inventive sustained release sm-active agent delivery system coating is also the fluid contacting surface, and is biocompatible and blood compatible.

### VIII. Exemplification

The invention now being generally described, it will be more readily understood by reference to the following examples, which are included merely for purposes of illustration of certain embodiments and embodiments of the present invention, and are not intended to limit the invention.

### EXAMPLE 1

### Transduction of Heat Shock Protein (HSP20) Phosphopeptides Alters Cytoskeletal Dynamics

It has previously been shown that transducible phosphopeptide analogs of HSP20 have physiological activity for relaxing smooth muscle in various tissues, including porcine coronary artery (Flynn et al., 2003, FASEB J. 17:1358) and bovine carotid artery (Woodrum et al., 2003, J. Vasc. Surg. 37:74). In addition, rat mesangial cells overexpressing HSP20 were refractory to serum-induced contraction, as demonstrated by wrinkle formation on a silicone polymer substrate (Woodrum et al., 2003, J. Vasc. Surg. 37:74).

The 14-3-3 proteins are thought to be general biochemical regulators because they are involved with many cellular functions and have a broad range of ligands, such as receptors, kinases, phosphatases, and docking molecules (Fu et al., 2000, Annu. Rev. Pharmacol. Toxicol. 40:617). For example, phosphorylated cofilin is stabilized by binding to 14-3-3 proteins (Gohla and Bokoch, 2002, Curr. Biol. 12:1704; Birkenfeld et al., 2003, Biochem. J. 369:45). Phosphorylated cofilin is inactive; however, when dephosphorylated by the slingshot family of phosphatases, cofilin catalyzes the depolymerization of actin (Niwa et al., 2002, Cell 108:233) and thus causes reorganization of the cytoskeleton.

To determine if the pHSP20 peptide binds to 14-3-3, pull-down experiments were conducted with pHSP20 and its analogues, aHSP20 and scrHSP20, linked to NHS activated Affigel 10 beads. As an additional control, activated beads were also reacted with ethanolamine. Each of the bead-bound peptide samples and the ethanolamine control were then separately incubated for 1.5 h at 4° C. with a whole cell lysate derived from HEK293 cells. After thoroughly washing the beads, each of the four sets of beads was eluted with a 100 μM solution of the free peptide corresponding to the one immobilized on the beads; the ethanolamine control beads were eluted with pHSP20. Approximately 15% of the eluate volume was run on an SDS-PAGE gel, while the remainder of the eluate was precipitated with ethanol and then analyzed by a 2D-LC shotgun MS method (Washburn et al., 2001, Nat. Biotechnol. 19:242).

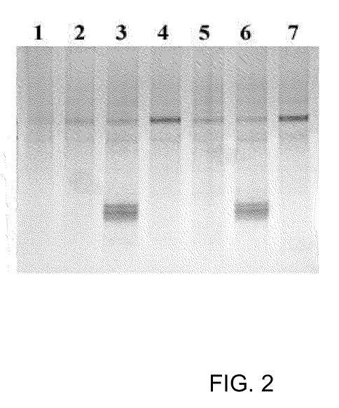

The immobilized pHSP20 lanes (FIG. 2) exhibit a diffuse set of bands at approximately 30 kDa; these bands are not evident in any of the controls. MS analysis of the various pull-down samples indicated that the only proteins that were identified with high confidence in the pHSP20 pull-down were various isoforms of 14-3-3 (Table 1). These isoforms of 14-3-3 were not associated with nonphosphorylated or scrambled peptide analogues.

TABLE 1
MS analysis of the pull-down samples.

| | | | # of peptides |
|---|---|---|---|
| Protein Name | Reference | Mascot Score | found |
| 14-3-3 epsilon | gi\|5803225 | 339 | 7 |
| 14-3-3 gamma | gi\|21464101 | 226 | 5 |
| 14-3-3 zeta | gi\|4507953 | 182 | 5 |
| 14-3-3 beta | gi\|4507949 | 162 | 5 |
| 14-3-3 eta | gi\|4507951 | 142 | 4 |
| 78 kDa gastrin- | gi\|595267 | 72 | 2 |
| binding protein | | | |
| Fibroblast-activating | gi\|539588 | 59 | 1 |
| factor 32K precursor | | | |
| DNA-activated protein | gi\|1362789 | 52 | 2 |
| kinase catalytic subunit | | | |
| Sequestosome 1 | gi\|4505571 | 51 | 1 |

Taken together, these data suggest that small peptides containing short sequences or motifs surrounding a phosphorylation site can have profound effects on cellular biology. Since these peptides have little or no predicted tertiary structure, these peptide motifs are likely altering cellular function through changes in protein-protein interactions. In the case of phosphorylated HSP20, these data suggest that the motif surrounding the phosphorylation site binds to 14-3-3 proteins. Such binding could increase the pool of unbound cofilin, leading to its dephosphorylation and activation as an actin depolymerizating protein.

### Materials and Methods

### 1. Peptide Synthesis and Purification

Peptides were synthesized using standard f-moc chemistry and purified using high performance liquid chromatography (HPLC) by Bio-Synthesis (Lewisville, Tex.). Fluorescent peptides were synthesized with a fluorescein isothiocyanate (FITC) labeled on the N terminus, using β-alanine as a linker.

### 2. Immobilization of the Peptides to Affigel 10 Beads

The N-terminal amino group of the peptides was utilized for the immobilization to N-hydroxysuccinimide activated Affi-Gel 10 beads (Biorad, Hercules, Calif.). For the immobilization, 60 μg of each peptide, dissolved in dimethylformamide (DMF), was incubated for 4 h with 100 μl beads and 0.14 μmol triethylamine (Sigma, St. Louis, Mo.). The final volume during the immobilization was 400 μl. After the incubation, the beads were washed extensively with DMF, and the remaining active groups were blocked by an over night incubation with 1 M ethanolamine (Sigma, St. Louis, Mo.). During the peptide synthesis the E-amino group of the lysine was protected with an ivDde protecting group. After immobilization the peptide was deprotected by incubation with 2% Hydrazine in DMF for 5 min, three times. The release of the ivDde group was monitored by measuring the absorption at 290 nm. The beads were then washed extensively with DMF and stored at 4° C.

### 3. Pull-Down Assay

A pull-down assay was conducted as described by Peltier, et al., Int. J. Mass Spectrometry, 2004, 238:119-130. Briefly, each set of beads (10 μl), on which the peptides had been immobilized, was separately incubated with 2 mg of HEK-293 cell lysate. The protein concentration in the cell lysate was approximately 5 mg/ml during the incubation. The beads were incubated with the lysate for 1.5 h at 4° C. and then washed 5 times with 1 ml washing buffer (20 mM HEPES, 10% Glycerol, 0.1% NP40, 250 mM NaCl, pH 7.0). Specifically bound proteins were eluted with 50 μl of wash buffer containing 100 μM of the free peptide, corresponding to the one immobilized on the beads. A sample (7 μl) of the eluate was used for analysis by SDS-PAGE, while the remaining eluate was precipitated by mixing with a 3-fold volume of ethanol and incubation for 12 h at −20° C. The precipitated samples were then submitted to 2D LC-MS/MS analysis (Zhen et al., 2004, J. Am. Soc. Mass Spectrometry 15: 803-822).

### 4. In-Solution Trypsin Digestion

The proteins in the pull-down samples were denatured in 8 M Urea/0.2 M NH₄HCO₃, and then reduced with 7.5 mM dithiothreitol at 60° C., and finally, alkylated with 15 mM of iodoacetamide. The solution was diluted to a final concentration of 2 M urea using de-ionized water (Milli-Q, Millipore, Bedford, Mass.) as the diluent, and trypsin (Promega, Madison, Wis.) was added to the sample at a protein/enzyme ratio of 20:1 by weight. The digestion was allowed to proceed at 37° C. for at least 2 hrs. The digested samples were split into two equal fractions for analysis by LC-MALDI-MS/MS and ESI-LC-MS/MS.

### 5. Strong Cation-Exchange Fractionation

The tryptic peptides were desalted with a peptide MicroTrap cartridge (Michrom BioSciences, Auburn, Calif.) and then loaded onto a Vydac 400VHP series strong cation-exchange (CEX) column (0.3×50 mm) (Grace Vydac, Hesperia, Calif.). The separation was done using an Agilent 1100 series binary pump with 0.5% acetic acid/20% acetonitrile (ACN) as buffer A, and 250 mM ammonium acetate in 0.5% acetic acid/20% ACN as buffer B. The CEX effluent was collected using a Probot micro fraction collector (LC-Packings, Sunnyvale, Calif.). Samples to be analyzed by LC-MALDI-MS/MS were separated into two CEX fractions, while those for LC-ESI-MS/MS were separated into 6 CEX fractions.

### 6. LC-MALDI-MS/MS

For those samples to be analyzed by MALDI-MS/MS, the peptides in the CEX fractions were further separated on a 75 μm×150 mm reverse-phase HPLC column (Dionex, Sunnyvale, Calif.). The samples were injected using a Famous autosampler and a Switchos II system (Dionex), and the HPLC gradient was controlled by an Ultimate system (Dionex). Solvent A was 0.1% TFA, and solvent B was 0.1% TFA/100% ACN. The flow rate was 250 nl/min. The HPLC eluate was mixed directly with MALDI matrix, at a flow rate of 800 nl/min, before being deposited on a bar-coded blank MALDI plate (Applied Biosystems), using a Probot (Dionex) micro fraction collector. The MALDI matrix was made up at a concentration of 3 mg/ml of alpha-cyano-4-hydroxyl-cinnaminic acid (CHCA) in 70% ACN. Spots were deposited every 20 seconds and a total of 144 spots were collected in a 12×12 array for each HPLC run. The samples on the MALDI plates were analyzed using a 4700 Proteomics Analyzer MALDI-TOF/TOF (Applied Biosystems, Foster City, Calif.). MS spectra were recorded for each spot, and MS/MS spectra were recorded for ions that passed the specified threshold criteria.

### 7. LC-ESI-MS/MS

ESI-MS/MS was performed using an LCQ Deca XP instrument (ThermoFinnigan, San Jose, Calif.) equipped with a custom built ESI source. An identical reverse phase HPLC system as described above for the LC-MALDI set-up was also used to perform the LC-ESI-MS/MS experiments. Solvent A was 0.5% acetic acid/2% ACN, and solvent B was 0.5% acetic acid/100% ACN. Data was acquired in a data dependent mode using 1 MS scan followed by 3 MS/MS scans of the three most abundant peaks in each MS scan, unless they were excluded by a dynamic exclusion window of 3 min.

### 8. MS Data Analysis

Data obtained from TOF/TOF and LCQ were searched using Mascot (Matrix Sciences, London, UK) as the search engine against the human subset of proteins in the NCBInr protein sequence database (Database version as of Oct. 10, 2003). The peak lists generated from the separately analyzed CEX fractions were merged prior to being submitted to the Mascot server. The two result files generated for the same sample from TOF/TOF and LCQ analyses were further merged into one file using custom software developed in house. The proteins identified in the pull-down samples derived from the various controls, were merged and used as a background subtraction list for the pHSP20 peptide pull-down. Only the proteins that remained after subtraction are reported in the results. Generally, proteins that have been identified from multiple peptides and have a Mascot protein score above 100 are considered confidently identified results.

### EXAMPLE 2

### Determination of the Binding Between Various Truncated Versions of the pHSP20 (Phosphorylated HSP20) Peptide and the 14-3-3 Gamma Isoform

To determine the relative binding affinity between various truncated versions of the pHSP20 (phosphorylated HSP20) peptide and the 14-3-3 gamma isoform, Applicants employed Biacore binding assays and the experiments were set up as follows. The pHSP20 peptide used in the original experiments (e.g., WLRRApSAPLPGLSK) was immobilized to a Biacore chip and competition experiments were performed by flowing a 14-3-3 gamma isoform protein over the chip in the presence of different pHSP20 truncation variants. The pHSP20 truncation variants included: a) pHSP20 (positive control); b) HSP20 (unphosphorylated pHSP20, negative control); c) RRApSAP (minimal 14-3-3 consensus binding sequence); d) WLRRApSAP; e) RRApSAPLP; f) RRApSAPLPGLS; g) WLRRApSAPLP.

Competition experiments were done whereby the only variable between experiments is the identity of the competing peptide. Hence, the relative ability of each peptide to compete with the original pHSP20 peptide should correlate inversely with the binding constant of each peptide for 14-3-3 gamma.

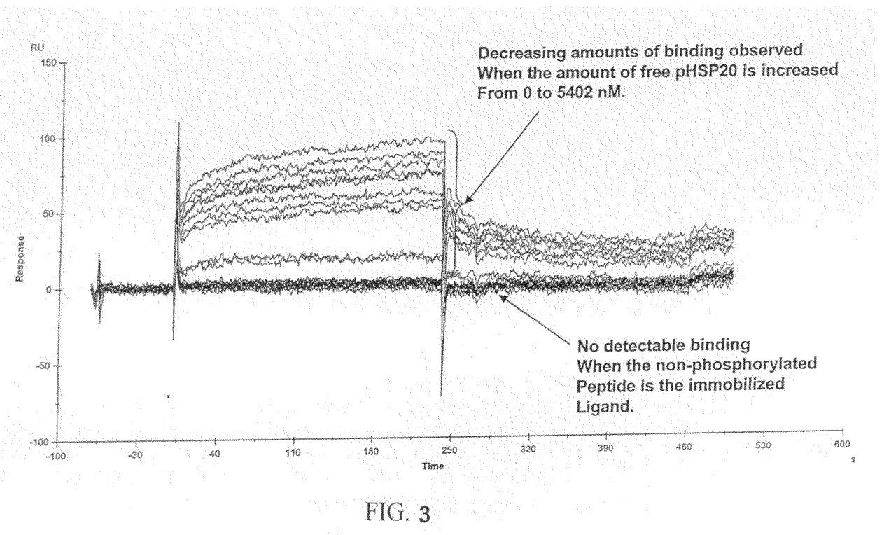

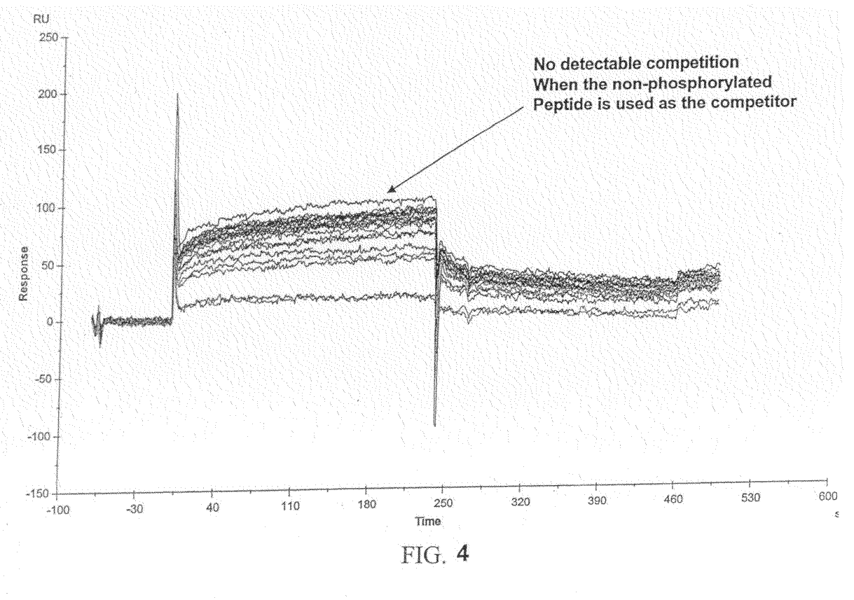

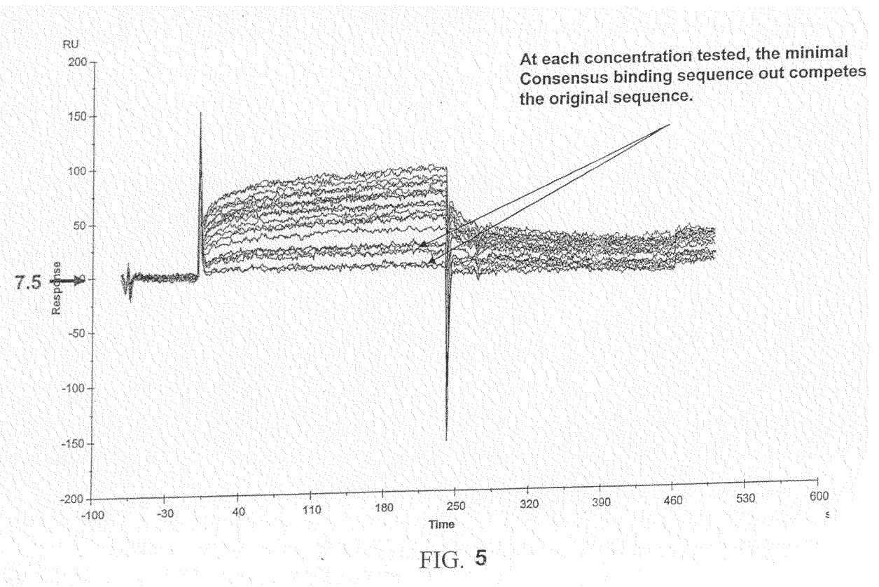

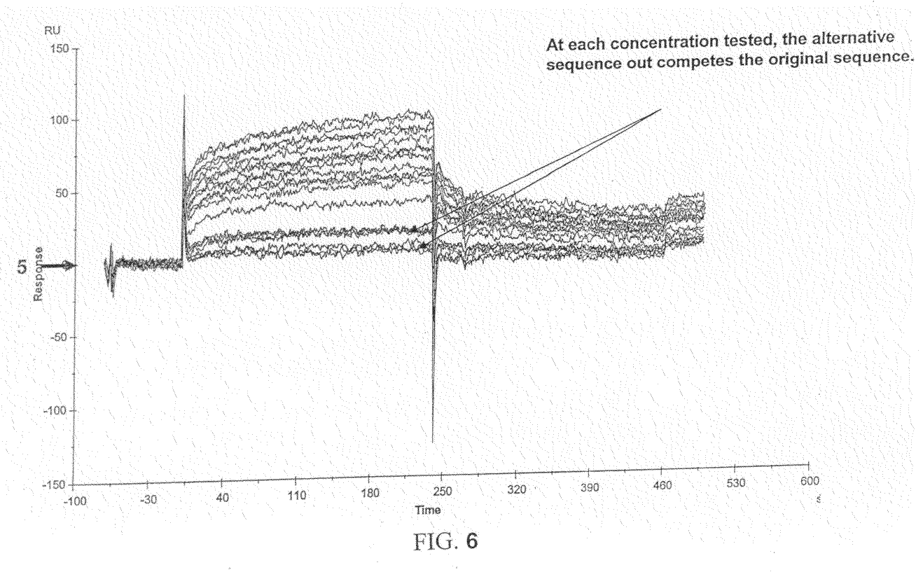

The results are shown in FIGS. 3-6. The minimal consensus 14-3-3γ-binding sequence was RRApSAP and it competed better than the original pHSP20 sequence (WLRRApSAPLPGLSK) for binding to 14-3-3γ. Hence, the binding constant between 14-3-3 gamma and the minimal consensus sequence was lower than that for the original pHSP20 peptide sequence (K_{D}≈6 nM, as determined by a Biacore experiment). In addition, this tight binding of the 14-3-3γ-binding consensus sequence to pHSP20 was unaffected by additional N-terminal residues (WL-) but was severely reduced by additional C-terminal residues (e.g., -LP or -LPGLS). However, this negative effect of the C-terminal residues was eliminated if the N-terminal residues were added back. This data suggests that a fluorophore can be added to the N-terminus of a peptide when used for Fluorescence Polarization experiments.

Taken together, the peptides RRApSAP and WLRRApSAP represent peptides which have a higher binding affinity to 14-3-3 gamma than the original pHSP20 peptide does. It remains to be determined what specificity each peptide has for the various 14-3-3 isoform. It may be that the selectivity of binding with the 14-3-3 gamma isoform is encoded in the amino acids that flank the minimal consensus binding sequence.

### EXAMPLE 3

### Determination of the Binding Between the pHSP20 Peptide and Various Versions and Isoforms of the 14-3-3

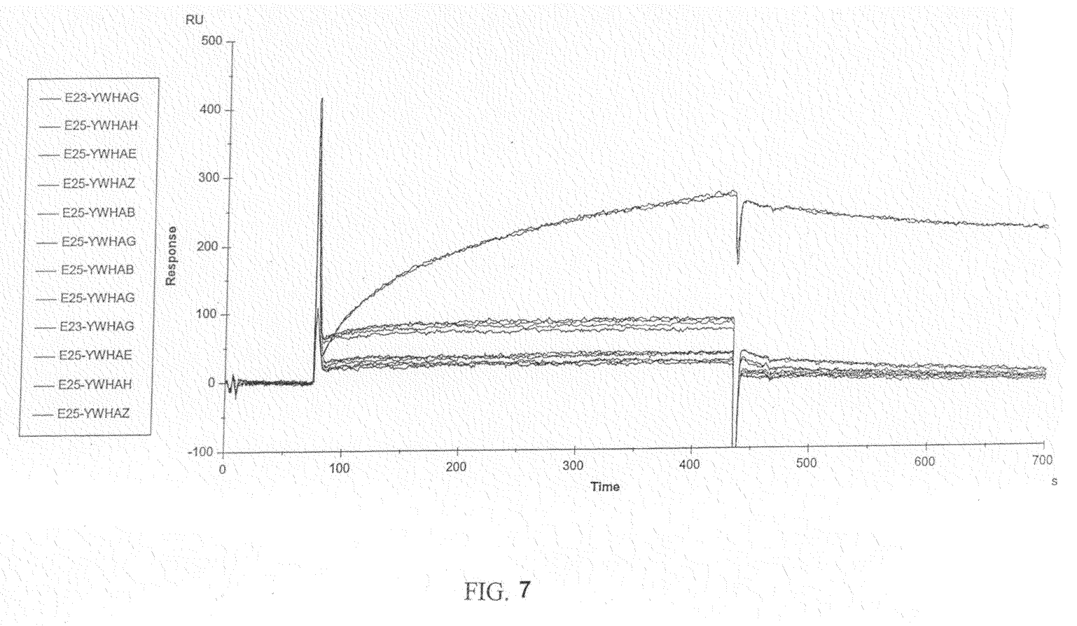

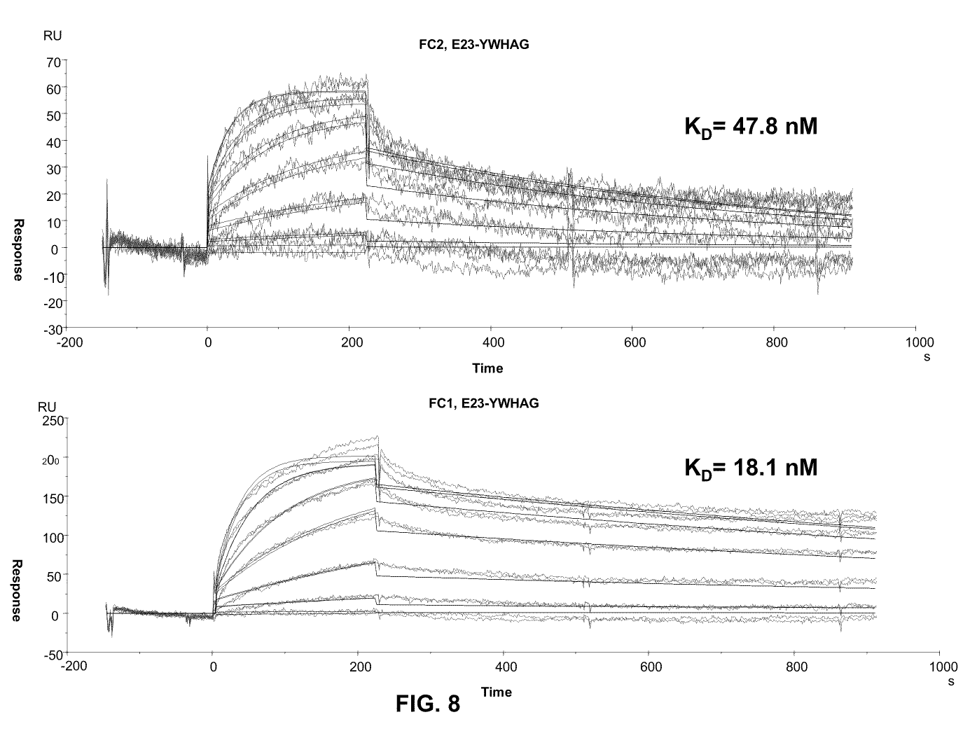

To further determine binding specificity of the pHSP20 peptide for each 14-3-3 isoform, similar binding experiments were carried out with each 14-3-3 isoform. Various 14-3-3 isoforms were either tagged with GST-His (referred to as “E23”) or tagged with Biotin-His (referred to as “E25”). These E23-tagged or E25-tagged 14-3-3 proteins were used in the Biacore experiments shown in FIGS. 2, 3, 4, 7 and 8. The E23-14-3-3 proteins (tagged with GST-His) bound much better than the E25-14-3-3 proteins (tagged with Biotin-His). Similar effects were detected by using a version of YWHAG (also referred to as 14-3-3γ) in which the GST tag was proteolytically removed. However, both tag-systems showed the same isoform specificity for the pHSP20 peptide (YWHAG>YWHAH>YWHAE=YWAHB=YWHAZ). It is believed that the GST either has a chaperone or stabilizing effect on YWHAG or it promotes the formation of dimers of YWHAG which could have different binding properties to pHSP20. Further experiments are planned to elucidate this discrepancy.

To determine the binding constant for the pHSP20 peptide, the E23 and E25-14-3-3 proteins were used in the Biacore experiments. For the E25-YWHAG a K_{d} of 25 nM to 1.3 μM was determined in several experiments However, due to the low signal of the E25-YWHAG compared to E23-YWHAG the quality of the fit is not as good and it is therefore not surprising that the estimated K_{d} varies so much. For the E23-YWAG a much tighter K_{d} of 5-50 nM was determined in several experiments. Certain experimental conditions could alter the absolute value of the K_{d}. For example, a high immobilization level of peptide on the chip can cause rebinding effects. Therefore, the kinetics for the pHSP20/14-3-3γ interaction may be in the high nM range

In sum, one of the goals of these studies is to find the minimal peptide that not only binds as well or better than the original pHSP20 peptide sequence, but also has binding specificity for a specific isoform of the 14-3-3 (e.g., the 14-3-3 gamma isoform). Moreover, similar binding experiments can be carried out to determine the binding constant using the Biacore instrument. These experiments can be used to confirm the implied relative affinities determined from the competition experiments as described above in Example 2.

### EXAMPLE 4

### Dose Response for Compounds Represented by General Formula I in Fluorescence Polarization Assay

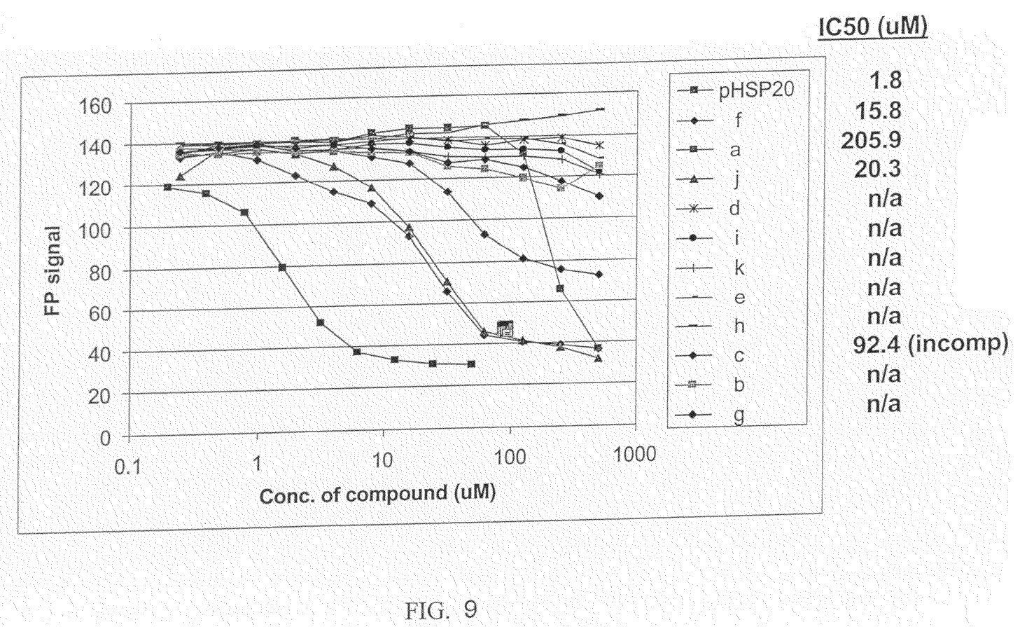

The ability of compounds (a)-(k) to inhibit the interaction between 14-3-3γ and pHSP20 was determined by a fluorescence polarization (FP) assay. Fluorescence Polarization measurements were made on samples arrayed in black 384-well plates (Greiner Bio-One) by using an Envision plate reader (Perkin-Elmer; excitation wavelength 480 nm and observed emission wavelength 535 nm) to monitor the interaction between the 14-3-3 gamma protein and an N-terminal 6-carboxy-fluorescein-labeled pHSP20 peptide with the amino acid sequence WLRRApSAP. The initial screen for inhibitors examined approximately 50,000 compounds in the DiverSet library (ChemBridge, San Diego). Initial compound screening was carried out by first mixing peptide and individual compounds followed by addition of protein to a final volume of 15 ul. The final concentrations of each component were: 57.4 nM peptide, 10 uM compound, 5% DMSO, 1.5 uM 14-3-3 gamma, 0.01 M HEPES pH 7.4, 0.15 M NaCl, 3 mM EDTA, 0.005% Tween 20, 10 mM MnCl₂, 9.33 mM Tris (7.5), and 0.0093% NaN₃. Several of the compounds, particularly (a), (f) and (j) were able to largely inhibit interaction between 14-3-3γ and pHSP20 in a dose-responsive manner. The results of the assay are shown in FIG. 9. The competitive inhibition of the interaction between 14-3-3γ and pHSP20 caused by an unlabelled phosphorylated pHSP20 peptide is shown as a control.

### EXAMPLE 5

### Dose Response for a Compound Represented by General Formula III in Fluorescence Polarization Assay

The ability of compounds (l) and (m) to inhibit the interaction between 14-3-3γ and pHSP20 was determined by a fluorescence polarization (FP) assay as described in Example 4, except that the compound was measured at multiple concentrations.

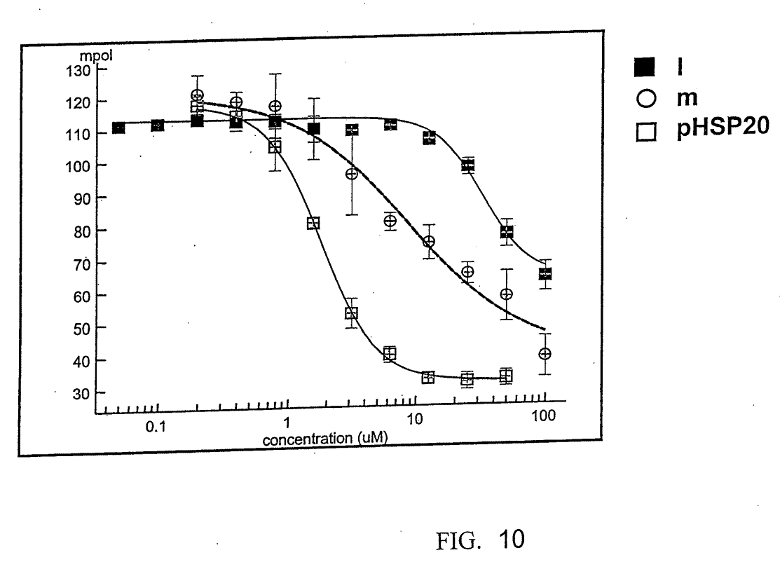

The results of the assay are shown in FIG. 10. Compound (l) was found to have an IC₅₀ of about 32 μM. Compound (m) was found to have an IC₅₀ of about 13.5 μM, while the control peptide pHSP20 had an IC₅₀ of about 2 μM.

### EXAMPLE 6

### Compounds Represented by General Formula IV Cause Dilation of Bovine Coronary Artery Rings

Bovine coronary artery rings were treated with compositions containing only cyclodextrin or formulations of cyclodextrin in combination with a pHSP20 peptide, compound (m) or compound (n). The contraction of the rings was measured over time after treatment with serotonin and one of the formulations above. Each of the formulations was tested at several concentrations, with the exception of the pHSP20 peptide.

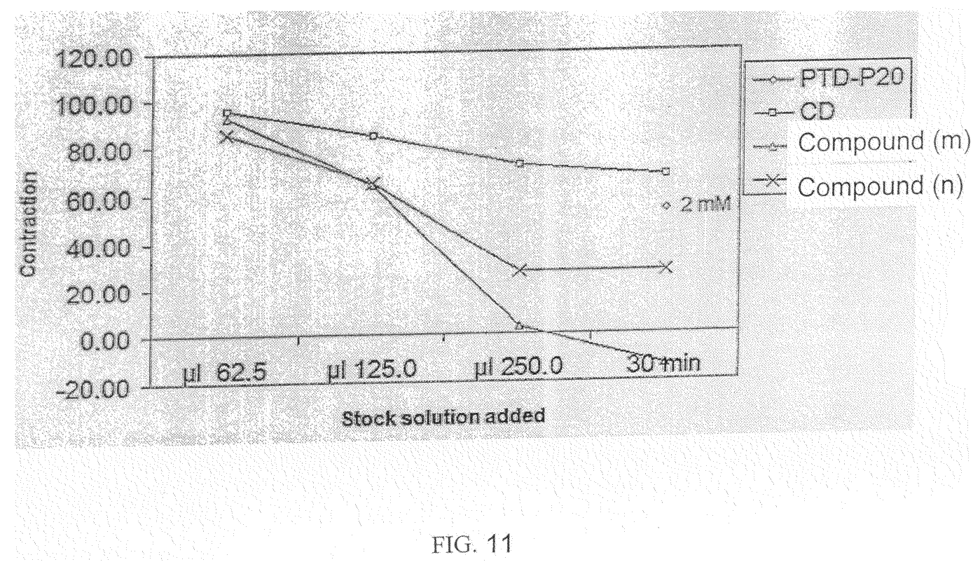

The percentage contraction of the rings at 30 minutes after treatment (10 minutes for the pHSP20 peptide) is shown in FIG. 11. Both compounds (m) and (n) had a vasodilatory effect on the rings. The effect of compounds (m) and (n) began to wear off about 20-25 minutes after addition, although no significant decrease in the vasodilatory effect was seen at 40 minutes after treatment. The vasodilatory effect of compounds (m) and (n) is longer than that of the pHSP20 peptide in this model.

### EXAMPLE 7

### Spontaneous Bead Motion Caused by Primary Human Airway Smooth Muscle Cells

Primary human airway smooth muscle cells were isolated from healthy individuals and cultured to passage 3-6. The cells were grown to confluence, and then serum deprived for 24 hours. The serum-deprived cells were plated on plastic wells coated with collagen.

After the cells were plated, RGD-coated microbeads were added. As described by Wang et al. Science 260:1124-1127, 1993, the microbeads become tightly anchored to the cell cytoskeleton. Consequently, the movement of the cells can be determined by monitoring the motion of the microbeads. In this assay, the rate of spontaneous bead motions (i.e., cell motion) depends upon the rate of reorganization of the actin cytoskeleton. Reduction of bead movement indicates stabilization of the cytoskeleton, whereas an increase in bead motion indicates an increase in actin depolymerization (An et al., J. Appl. Physiol. 96:1701-1713, 2004).

The position of each bead was recorded using video microscopy. The two-dimensional trajectory of bead motion are expressed as a mean square displacement (MSD) as a function of time:

$$M S D ( t ) = \frac{1}{N} {\sum }_{i = 1}^{N} {{r}_{i} ( t )}^{2} ,$$

where rᵢ(t) is the distance of the ith bead at time t relative to its position at time 0.

The spontaneous bead motion in each sample of cells was first measured for 5 minutes. A test compound (nothing for the time control) was then added and the sample was incubated for 30 minutes (10-15 minutes for non-peptidyl compounds of the invention). Spontaneous bead motions were then measured for another 5 minutes.

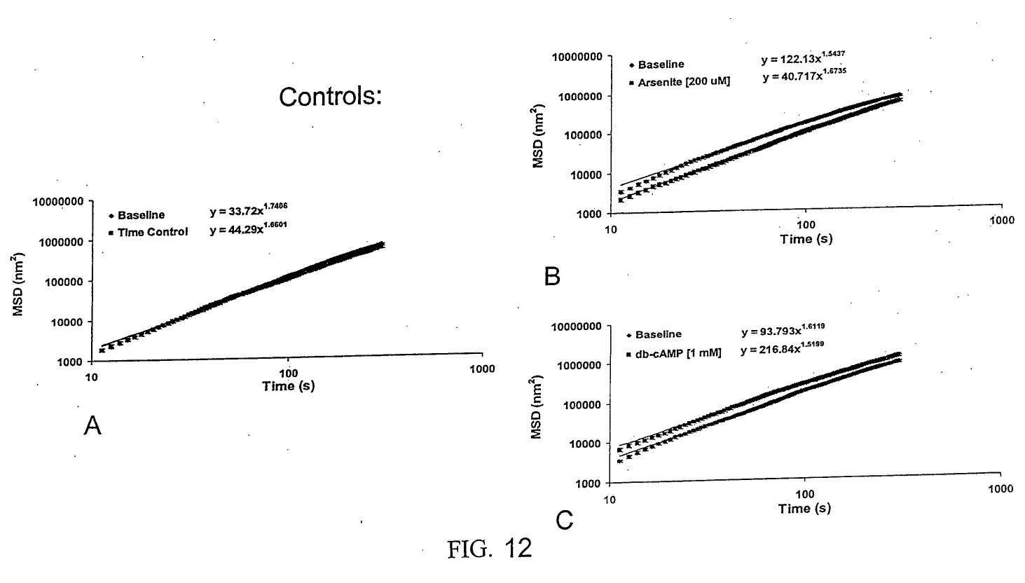

Three control runs were conducted with nothing (the time control), sodium arsenite (negative control, promotes phosphorylation of HSP27) and dibutyl-cyclic adenosine monophospate (positive control, destabilizes cytoskeleton). The MSD plots for the time control and samples treated with 200 μM sodium arsenite and 1 mM db-cAMP are shown in FIGS. 12A-C, respectively. As expected, the time control shows no change, arsenite causes a decrease in bead movement and db-cAMP increased the bead motion.

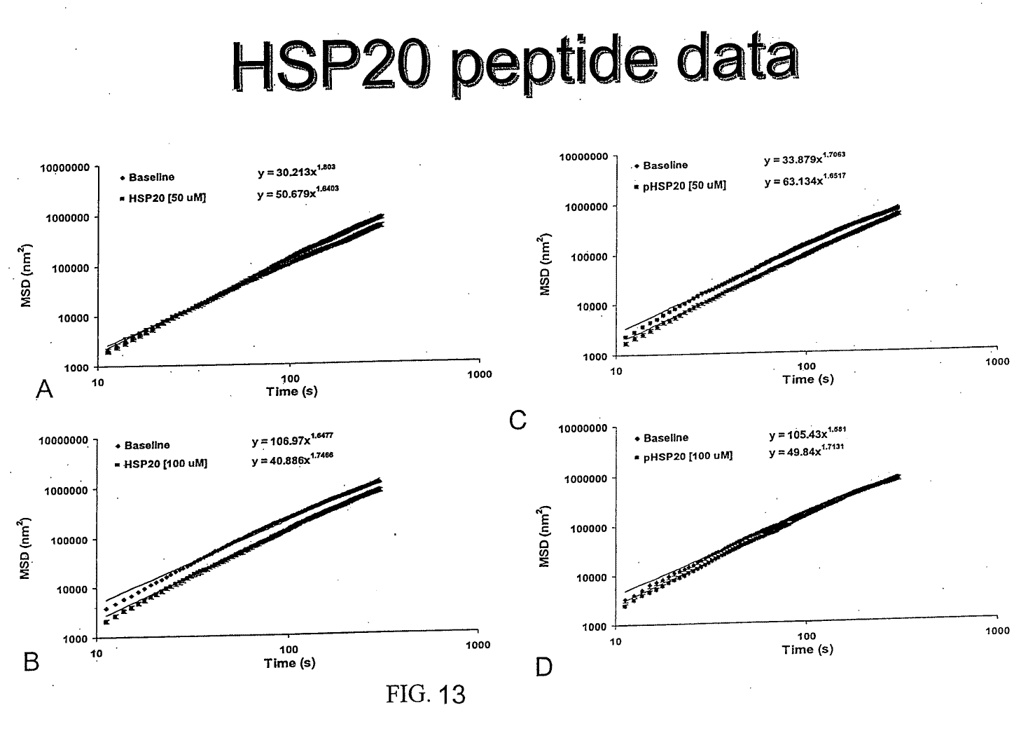

The cells were also treated with various concentrations of phosphorylated and non-phosphorylated PTD-HSP20 peptide. The MSD plots are shown FIGS. 13A-D. The non-phosphorylated PTD-HSP20 had almost no effect at 50 μM, while the phosphorylated peptide caused an increase in bead movement at the same concentration. At an increased concentration, 100 μM, both the phosphorylated and non-phosphorylated PTD-HSP20 peptides decreased the amount of bead movement. One explanation for this phenomenon, albeit not verified, is that the peptide is toxic to cells at the 100 μM concentration.

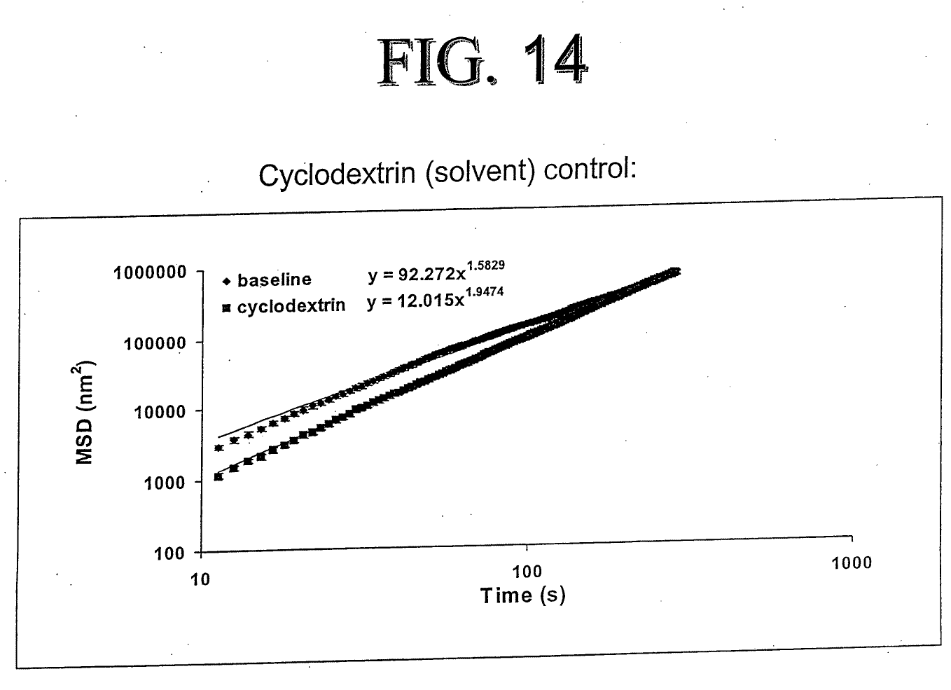

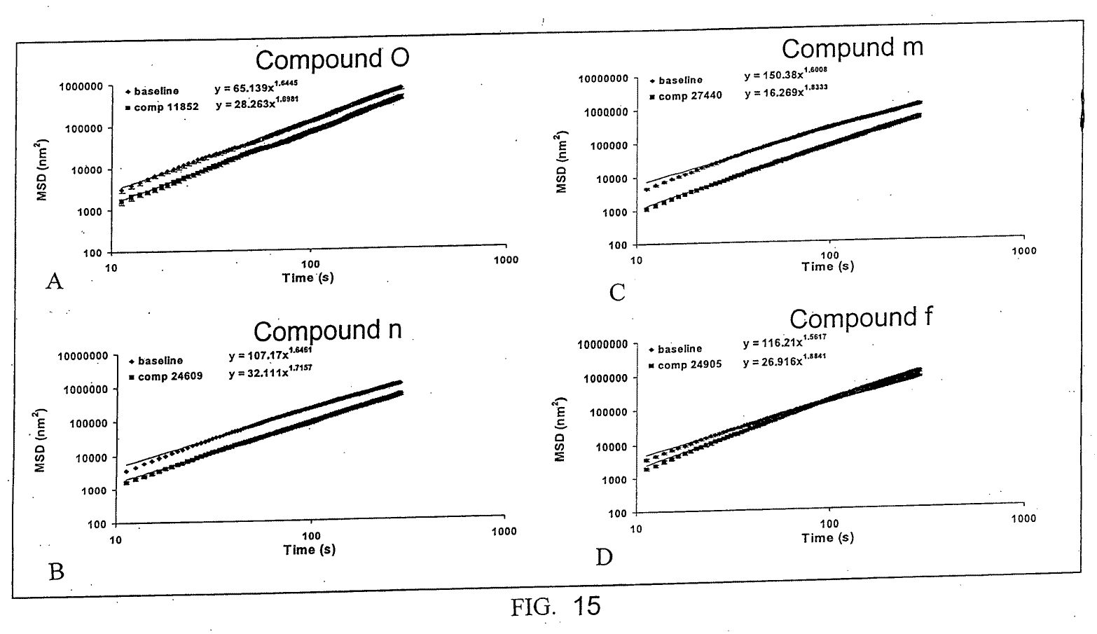

The non-peptidyl compounds of the invention had to be formulated with 4% cyclodextrin, due to their low water solubility. The MSD plot of the cyclodextrin control is shown in FIG. 14. The MSD plots of non-peptidyl compounds (o), (m), (n) and (f) are shown in FIGS. 15A-D, respectively. The cyclodextrin control had an effect on bead motion, namely that motion was decreased. As a result, the effect of the non-peptidyl compounds is somewhat masked by the cyclodextrin. Although compound (f) clearly increases bead motion, the results are not conclusive as to what effect the other three compounds have.

### EXAMPLE 8

### Magnetic Twisting Cytometry

Primary human airway smooth muscle cells were isolated from healthy individuals and cultured to passage 3-6. The cells were grown to confluence, and then serum deprived for 24 hours. The serum-deprived cells were plated on plastic wells coated with collagen. After the cells were plated, RGD-coated microbeads were added. As described by Wang et al. Science 260:1124-1127, 1993, the microbeads become tightly anchored to the cell cytoskeleton. Consequently, the movement of the cells can be determined by monitoring the motion of the microbeads.

Once the cells were attached to the microbeads, the beads were magnetized using a magnetizing coil and twisted using a twisting coil, which generates an oscillating magnetic field at 0.7 Hz. The bead displacement is measured using video microscopy at 83 ms intervals. The amplitude of bead displacement is dependent on several factors, but generally the displacement is directly proportional to cell stiffness. The cell stiffness is expressed as a change of storage modulus (G′) over time (Maksym et al., J. Appl. Physiol. 89: 1619-1632, 2000).

The cell stiffness for control experiments and experiments using peptides was measured over 10 minutes. The peptides were added 1 minute after the start of the experiment. The cell stiffness for non-peptidyl compounds of the invention was measured for 1 minute, stopped, compound added and stiffness was measured for another 10 minutes.

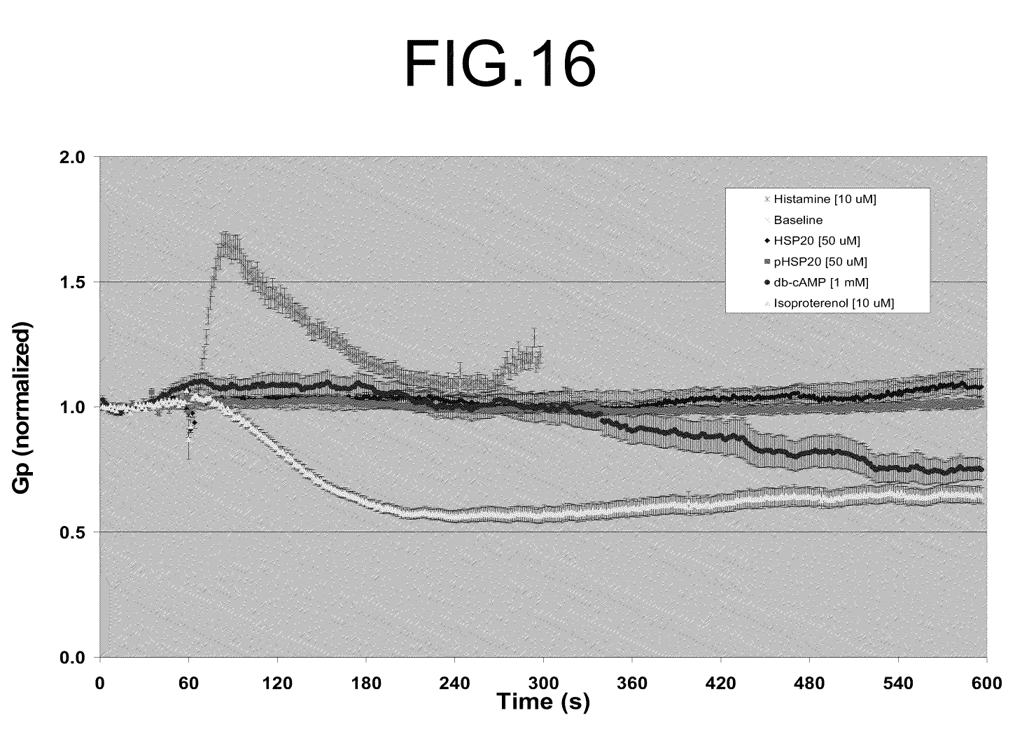

The change of storage modulus (cell stiffness) over time for the controls is shown in FIG. 16. In the controls, histamine (positive control) increased the cell stiffness, while isoproterenol and db-cAMP (negative controls) decreased the stiffness. At the concentration test, non-phosphorylated PTD-HSP20 peptide does not show a statistically significant difference from the baseline. In contrast, the phosphorylated peptide exhibited a statistically significant decrease in stiffness near the 10 minute time point.

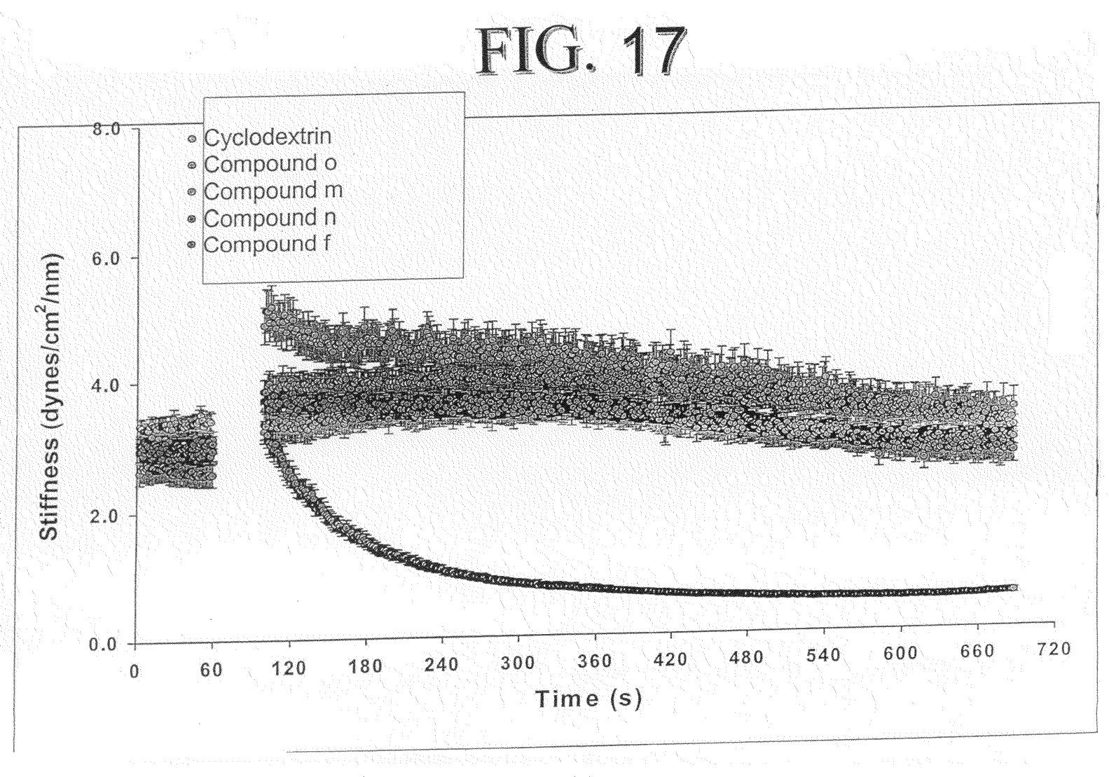

As in Example 7, cyclodextrin was required to solubilize the non-peptidyl compounds of the invention. FIG. 17 shows that the cyclodextrin control increased cell stiffness, while all compounds of the invention reduced cell stiffness compared to the cyclodextrin control. The effect of compound (f) was particular striking.

### INCORPORATION BY REFERENCE

All publications and patents mentioned herein are hereby incorporated by reference in their entirety as if each individual publication or patent was specifically and individually indicated to be incorporated by reference.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A composition for modulating smooth muscle contractility comprising an agent with a structure of Formula (III): 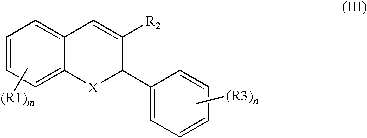
or a pharmaceutically acceptable salt thereof, wherein:
each R1 and R3 is independently selected from halogen, CF3, C1-6 alkyl, cycloalkyl, amino, hydroxyl, alkoxy, nitro, carboxy, carboxyesters, carboxamide and sulfonamide;
R2 is selected from nitro, carboxy, carboxyester, substituted carboxamide, and C1-6 alkyl;
X is selected from NH and O;
m is an integer from 0 to 4; and
n is an integer from 0 to 5;
or a structure of Formula (IV): 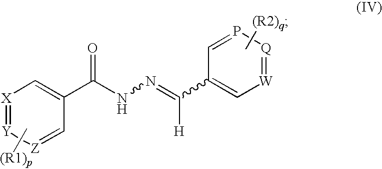
or a pharmaceutically acceptable salt thereof, wherein:
each R1 and R2 is independently selected from hydroxyl, C1-3 alkoxy, C4-6 cycloalkoxy, nitro, amino, acyl, carboxyl, carboxy ester, carboxamide, and sulfonamide;
X, Y, Z, P, Q, and W are independently selected from CH and N;
p is an integer from 0 to 5; and
q is an integer from 0 to 5.

2. A respiratory formulation comprising a composition of claim 1.

3. A metered dose aerosol dispenser containing an aerosol pharmaceutical composition for pulmonary or nasal delivery comprising a composition of claim 1.

4. A method for modulating smooth muscle contractility comprising administering a composition of claim 1.

5. A method for treating a patient suffering from the effects of vasoconstriction, vasospasms or restricted blood flow, comprising administering the composition of claim 1, wherein the agent enhances vasodilation.

6. A method for treating a patient suffering from bronchial constriction or bronchial spasm, comprising administering the composition of claim 1, wherein the agent enhances bronchial dilation.

7. A method for dilating bronchi in a patient, comprising administering the composition of claim 1, wherein the agent enhances bronchial dilation.

8. A method of inducing vasodilation to treat or prevent a vascontractive response or condition, comprising administering the composition of claim 1.

9. A method of increasing blood flow in the circulatory system of an animal comprising administering to said mammal the composition of claim 1.

10. A sustained release formulation comprising a polymer matrix and the composition of claim 1 dispersed in the polymer.

11. A medical device comprising:
(i) a substrate having a surface; and
(ii) a coating adhered to the surface, said coating comprising a polymer matrix including the composition of claim 1 dispersed therein in a manner that permits the agent to be eluted from the matrix under physiological conditions.

12. A coated device combination, comprising a medical device for implantation within a patient's body, said medical device having one or more surfaces coated with a polymer formulation including the composition of claim 1 in a manner that permits the coated surface to release the agent over a period of time when implanted in the patient.

13. An intraluminal medical device coated with a sustained release system comprising a biologically tolerated polymer and the composition of claim 1 dispersed in the polymer, said device having an interior surface and an exterior surface; said device having said system applied to at least a part of the interior surface, the exterior surface, or both.

14. A coating composition for use in delivering a medicament from the surface of a medical device positioned in vivo, the composition comprising a polymer matrix having an non-peptidyl agent that alters formation or stability of complexes including phosphorylated heat shock protein 20 (HSP20) and 14-3-3γ protein, or mimics the effect of HSP20 binding to the 14-3-3γ protein, which coating composition is provided in liquid or suspension form for application to the surface of said medical device by spraying and/or dipping the device in said composition.

15. A method for regulating contractility and/or tone of explanted vascular tissue, comprising contacting the explanted tissue in vitro with the composition of claim 1.

16. A method of identifying a candidate non-peptidyl therapeutic agent for modulating smooth muscle tone comprising:
(a) admixing a test agent, a 14-3-3 polypeptide, and a phosphorylated HSP20 polypeptide under conditions that, in the absence of the test agent, would permit interaction of the 14-3-3 and phosphorylated HSP20 polypeptides;
(b) determining if the test agent alters the interaction of the 14-3-3 and phosphorylated HSP20 polypeptides; and
(c) if the test agent alters the interaction of the 14-3-3 and phosphorylated HSP20 polypeptides, contacting the test agent with smooth muscle tissue and determining if the test agent alters the contractility and/or tone of the smooth muscle tissue.

17. A method of identifying a candidate non-peptidyl therapeutic agent for modulating smooth muscle tone comprising:
(a) admixing a test agent, a 14-3-3γ polypeptide and a cofilin polypeptide under conditions that, in the absence of the test agent, would permit interaction of the 14-3-3γ and cofilin polypeptides;
(b) determining if the test agent alters the interaction of the 14-3-3γ and cofilin polypeptides; and
(c) if the test agent alters the interaction of the 14-3-3γ and cofilin polypeptides, contacting the test agent with smooth muscle tissue and determining if the test agent alters the contractility and/or tone of smooth muscle tissue.
